(19) United States
(12) Patent Application Publication
(10) Pub. No.: US 2006/0015058 A1
(43) Pub. Date: Jan. 19, 2006

(54) Agents and methods for enhancement of transdermal transport

(76) Inventors: Scott C. Kellogg, Boston, MA (US); Shikha Barman, Bedford, MA (US); Lauren Roode, Cumberland, RI (US); Hannah Farnham, Sandwich, MA (US); Sean Moran, Norfolk, MA (US); Samir S. Mitragotri, Goleta, CA (US); Joseph Kost, Cambridge, MA (US); Nicholas F. Warner, Belmont, MA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/065,278

(22) Filed: Feb. 25, 2005

Related U.S. Application Data
(63) Continuation-in-part of application No. 10/974,963, filed on Oct. 28, 2004.
(63) Continuation-in-part of application No. 09/979,096, filed on Mar. 11, 2002.
(63) Continuation-in-part of application No. 09/868,442, filed on Jul. 24, 2001.
Application No. 09/868,442 is a continuation-in-part of application No. 09/227,623, filed on Jan. 8, 1999, now Pat. No. 6,190,315.
(60) Provisional application No. 60/112,953, filed on Dec. 18, 1998.
(60) Provisional application No. 60/142,941, filed on Jul. 12, 1999.
(60) Provisional application No. 60/142,950, filed on Jul. 12, 1999.
(60) Provisional application No. 60/142,951, filed on Jul. 12, 1999.
(60) Provisional application No. 60/142,975, filed on Jul. 12, 1999.
(60) Provisional application No. 60/070,813, filed on Jan. 8, 1998.

(51) Int. Cl. A61B 17/20; A61B 5/00

(52) U.S. Cl. 604/22; 600/573

(57) ABSTRACT

The invention according to an exemplary embodiment relates to a method for transporting a substance across a biological membrane comprising the steps of applying a delipidation agent to a portion of the biological membrane, applying a hydration agent to the portion of the biological membrane, sonicating the portion of the biological membrane, and transporting the substance across the biological membrane. The step of applying the delipidation agent may be carried out prior to or simultaneously with the step of applying the hydration agent. The hydration agent may be applied before, during, or after the sonication step. The methods according to exemplary embodiments of the invention can provide improved transdermal transport in applications such as continuous analyte extraction and analysis and transdermal delivery of drugs and vaccines.

The present application is a continuation-in-part of U.S. application Ser. No. 10/974,963, filed Oct. 28, 2004. The present application is also a continuation-in-part of U.S. application Ser. No. 09/979,096, which is a 371 of International Application No. PCT/US01/08489, filed Mar. 16, 2001. The present application is also a continuation-in-part of U.S. application Ser. No. 09/868,442, which is a 371 of International Application No. PCT/US99/30065, filed Dec. 17, 1999, which claims priority to the following five U.S. Provisional Applications: U.S. Provisional Application No. 60/112,953, filed Dec. 18, 1998; U.S. Provisional Application No. 60/142,941, filed Jul. 12, 1999; U.S. Provisional Application No. 60/142,950, filed Jul. 12, 1999; U.S. Provisional Application No. 60/142,951, filed Jul. 12, 1999; and U.S. Provisional Application No. 60/142,975, filed Jul. 12, 1999. U.S. application Ser. No. 09/868,442 is also a continuation-in-part of U.S. application Ser. No. 09/227,623, filed Jan. 8, 1999, now U.S. Pat. No. 6,190,315, which claims priority to U.S. Provisional Application No. 60/070,813, filed Jan. 8, 1998. The present application claims priority to all of the aforementioned applications. All of the aforementioned applications are incorporated by reference herein in their entireties.

### BACKGROUND OF THE INVENTION

1. Field of the Invention

The present invention relates to improvement of transdermal transport across a biological membrane by treating the biological membrane with a delipidation and/or a hydration agent. The present invention also relates to non-invasive sampling of body fluids, and, more particularly, to a system, method, and device for non-invasive body fluid sampling and analysis. The present invention also relates to transdermal delivery of small molecule drugs and biologics such as vaccines.

2. Description of the Related Art

Diabetics frequently prick their fingers and forearms to obtain blood in order to monitor their blood glucose concentration. This practice of using blood to perform frequent monitoring can be painful and inconvenient. New, less painful methods of sampling body fluids have been contemplated and disclosed. For example, these painless methods include the use of tiny needles, the use of iontophoresis, and the use of ultrasound to sample body fluid, such as blood and interstitial fluid.

It has been shown that the application of ultrasound can enhance skin permeability. Examples of such are disclosed in U.S. Pat. No. 4,767,402, U.S. Pat. No. 5,947,921, and U.S. Pat. No. 6,002,961, the disclosures of which are incorporated, by reference, in their entireties. Ultrasound may be applied to the stratum corneum via a coupling medium in order to disrupt the lipid bilayers through the action of cavitation and its bioacoustic effects. The disruption of stratum corneum, a barrier to transport, allows the enhanced diffusion of analyte, such as glucose or drugs, through, into, and out of the skin.

Transport of analytes and body fluids can be enhanced further by the action of a motive force. These motive forces include, inter alia, sonophoretic, iontophoretic, electromotive, pressure force, vacuum, electromagnetic motive, thermal force, magnetic force, chemomotive, capillary action, and osmotic. The use of active forces provides a means for obtaining fluid for subsequent analysis.

The application of a motive force before, during, and after making the skin permeable has been disclosed in U.S. Pat. No. 5,279,543, U.S. Pat. No. 5,722,397, U.S. Pat. No. 5,947,921, U.S. Pat. No. 6,002,961, and U.S. Pat. No. 6,009,343, the disclosures of which are incorporated by reference in their entireties. The purpose of using a motive force is to actively extract body fluid and its content out of the skin for the purpose of analysis. As mentioned, active forces, such as vacuum, sonophoresis, and electrosmotic forces, can create convective flow through the stratum corneum. Although these forces can be used for extraction of body fluids, there are certain limitations that may apply when the forces are applied to human skin. For example, a major limitation is the flow and volume of body fluid that can be transported across the stratum corneum. In general, high-pressure force is necessary in order to transport fluid across an enhanced permeable area of stratum corneum. The application of vacuum on skin for an extended period may cause physical separation of the epidermis from the dermis, resulting in bruises and blisters.

Another example of a limitation is the amount of energy that can be applied to the skin in order to create convective flow. Extraction of usable volume of body fluid has the potential to cause pain and skin damage with prolonged exposure to ultrasound. In a similar manner, electro-osmotic extraction of body fluid through stratum corneum has the potential to cause skin damage due the need to use high current density. It is evident that there are limitations to the use of the mentioned extraction methods when applied to human skin.

### SUMMARY OF THE INVENTION

A need has arisen for a system, method, and device for permeation of a biological membrane that reduces pain and/or discomfort yet facilitates permeation for transdermal extraction and analysis of analytes, transdermal drug delivery, and/or transdermal vaccination.

According to one embodiment of the invention, a hydrating agent can be applied to the biological membrane before and/or during and/or after sonication to enhance transdermal transport.

According to another embodiment, the invention relates to a method for transporting a substance across a biological membrane comprising the steps of applying a delipidation agent to a portion of the biological membrane, applying a hydration agent to the portion of the biological membrane, sonicating the portion of the biological membrane, and transporting the substance across the biological membrane. The step of applying the delipidation agent may be carried out prior to or simultaneously with the step of applying the hydration agent. The hydration agent may be applied to the biological membrane before, during or after the sonication step. The methods according to exemplary embodiments of the invention can provide improved transdermal transport in applications such as continuous analyte extraction and analysis and transdermal delivery of drugs and vaccines.

According to another embodiment, the invention relates to a method of preparing a biological membrane for sonication comprising treating the biological membrane with a solution. The method of preparing a biological membrane for sonication comprises steps of treating the biological membrane with a solution comprising alcohol, Lippo Gel, bile salt, or glycerol prior to sonication.

According to another embodiment, the invention relates to a method of transporting a substance across a biological membrane. The method of transporting a substance across a biological membrane comprises steps of providing a biological membrane exposed to a first pressure; sonicating a portion of the biological membrane; changing the pressure over the portion of the biological membrane to a second pressure after sonicating the portion of the biological membrane; and transporting the substance across the biological membrane.

According to another embodiment, the invention relates to a method for sampling and analysis of an analyte from a body fluid in a patient. The method for sampling and analysis of an analyte from a body fluid in a patient comprises the steps of increasing a permeability level of an area of a biological membrane of the patient; applying a transport force to the area to extract the analyte from the body fluid in the patient through the area and into a medium; continuously reacting the analyte in the medium to produce an electrical signal at an electrode adjacent to the medium, the electrical signal representing a rate of reaction of the analyte; and calculating an analyte concentration in the body fluid in the patient based on the electrical signal.

According to another embodiment, the invention relates to a transdermal analyte monitoring system. The transdermal analyte monitoring system comprises a sensor body; a medium supported by the sensor body and adapted to interface with a biological membrane, wherein the medium is adapted to prevent accumulation of analytes and analyte indicators during operation; and an electrode adapted to detect the presence of an analyte within the medium.

According to another embodiment, the invention relates to a method for transdermal analyte monitoring. The method for transdermal analyte monitoring comprises the steps of providing a sensor body, a medium and an electrode; positioning the medium adjacent to the surface of a biological membrane; and monitoring a flux of the analyte through the biological membrane using the electrode.

According to another embodiment, the invention relates to a transdermal analyte monitoring system. The transdermal analyte monitoring system comprises a medium adapted to interface with a biological membrane and to receive an analyte from the biological membrane; and an electrode assembly comprising a plurality of electrodes; wherein the medium is adapted to react continuously with the analyte; and wherein an electrical signal is detected by the electrode assembly, and the electrical signal correlates to an analyte value.

According to another embodiment, the invention relates to a transdermal analyte monitoring system. The transdermal analyte monitoring system comprises a medium adapted to interface with a biological membrane and to receive an analyte from the biological membrane; and a sensor comprising an electrode assembly, the electrode assembly comprising a plurality of electrodes; wherein the medium is adapted to react continuously with the analyte, an electrical signal is detected by the electrode assembly, and the electrical signal correlates to an analyte value.

According to another embodiment, the invention relates to a method for monitoring an analyte. The method for monitoring an analyte comprises positioning a medium with respect to a biological membrane such that the medium can receive an analyte from the biological membrane; coupling an electrode assembly to the medium, the electrode assembly comprising a plurality of electrodes; and continuously reacting the analyte with the medium; wherein an electrical signal is detected by the electrode assembly, and the electrical signal correlates to an analyte value.

According to another embodiment, the invention relates to a cartridge for use with a sonication device. The cartridge for use with a sonication device comprises a cartridge body adapted for insertion into the sonicating device; and a chamber within the cartridge body.

According to another embodiment, the invention relates to a system for sampling and analysis of an analyte from a body fluid in a patient. The system for sampling and analysis of an analyte from a body fluid in a patient comprises an ultrasonic applicator that applies ultrasound to an area of a biological membrane of the patient; a medium comprising a substance that reacts with the analyte; an electrode positioned adjacent to the medium, the electrode being adapted to receive an electrical signal produced by the reaction of the analyte with the substance in the medium, the electrical signal representing a rate of reaction of the analyte with the substance; and a processor electrically connected to the electrode; wherein the processor is programmed to continuously calculate a concentration of the analyte in the body fluid of the patient based on the electrical signal.

According to another embodiment, the invention relates to a method for sampling and analysis of an analyte from body fluid in a patient. The method comprises increasing the permeability of an area of a biological membrane of the patient, applying a transport force to the area to extract the analyte from the body fluid in the patient through the area and into a medium, continuously reacting the analyte in the medium to produce an electrical signal at an electrode adjacent to the medium, the electrical signal representing a rate of reaction of the analyte, and calculating an analyte concentration in the body fluid in the patient based on the electrical signal.

The invention also relates to enhancing the permeability of a biological membrane, such as skin, buccal, and nails, for an extended period of time, and a method for extracting body fluid to perform blood, interstitial fluid, lymph, or other body fluid analyte monitoring in a discrete or continuous manner that is noninvasive and practical.

The area of biological membrane may be made permeable using ultrasound with controlled dosimetry. Extraction of body fluid may be performed on the area exposed to ultrasound using osmotic transport. The body fluid may be collected using a receiver. The receiver may be attached to the biological membrane in a form of a patch, a wearable reservoir, a membrane, an absorbent strip, a hydrogel, or an equivalent. The receiver may be analyzed for the presence of various analytes indicative of blood analytes. The analysis may comprise the use of electrochemical, biochemical, optical, fluorescence, absorbance, reflectance, Raman, magnetic, mass spectrometry, infra-red (IR) spectroscopy measurement methods and combinations thereof.

A system for non-invasive body fluid sampling and analysis is disclosed. According to one embodiment of the present invention, the system includes a controller that controls the generation of ultrasound; an ultrasonic applicator that applies the ultrasound to an area of biological membrane; a receiver that contacts the area of biological membrane and receives body fluid through and out of the area of biological membrane; and a meter that interacts with the receiver and detects the presence of at least one analyte in the body fluid in the receiver. The receiver may include a membrane and a medium, such as a hydrogel, a fluid, or a liquid, that is contained within the membrane.

A method for noninvasive body fluid sampling and analysis is disclosed. According to one embodiment of the present invention, the method includes the steps of (1) enhancing a permeability level of an area of biological membrane; (2) attaching a receiver to the area of biological membrane; (3) extracting an analyte through and out of the area of biological membrane; (4) collecting the body fluid in the receiver; and (5) determining a concentration of at least one analyte in the body fluid.

A device for noninvasive body fluid sampling and analysis is disclosed. According to one embodiment of the present invention, the device includes a receiver that is attached to an area of biological membrane with an enhanced permeability and receives body fluid through and out of the area of biological membrane, and a wearable meter that detects the presence of at least one analyte in the received body fluid and indicates a concentration of that analyte. The receiver may include a membrane and a medium, such as a hydrogel, a fluid, or a liquid, that is contained in the membrane. The meter may include a processor and a device that detects the presence of the analyte. The detecting device may include an electrochemical detector; a biochemical detector; a fluorescence detector; an absorbance detector; a reflectance detector; a Raman detector; a magnetic detector; a mass spectrometry detector; an IR spectroscopy detector; and combinations thereof.

According to one embodiment of the present invention, osmotic forces may be used to sample body fluid from and through a biological membrane in an on-demand manner. The osmotic agent in solution, gel, hydrogel, or other form may be applied to the ultrasound-treated biological membrane using a receiver, such as a thin liquid reservoir, whenever the concentration of an analyte needs to be determined for diagnosis and monitoring. The receiver may be attached to the biological membrane using an adhesive. The receiver may be attached to the biological membrane for a brief duration. The solution in the receiver may be subsequently removed and analyzed for the presence of analytes. In one embodiment, the receiver may be constructed in the form of a patch. The receiver may contain a hydrogel and osmotic agent. The receiver may combine the osmotic agent and the chemical reagents to detect the presence of the analyte. The reagents may allow the use of electrochemical, biochemical, optical, fluorescence, absorbance, reflectance, Raman, magnetic, mass spectrometry, infrared (IR) spectroscopy measurement methods and combinations thereof to be performed on the receiver.

In another embodiment, osmotic forces may be used to sample body fluid from or through a biological membrane in a periodic or a continuous manner. The osmotic agent in solution form may be applied to the ultrasound-treated biological membrane using a thin receiver, such as a thin liquid reservoir, whenever the concentration of analyte needs to be determined for diagnosis and monitoring. The receiver may be attached to biological membrane using an adhesive. In one embodiment, the receiver may be constructed in the form of a patch. The receiver may contain a hydrogel that contains the osmotic agent. The receiver may contain means for manipulating the intensity and duration of the osmotic force. The intensity of the osmotic force may be manipulated using electric field forces, magnetic field forces, electromagnetic field forces, biochemical reactions, chemicals, molarity adjustment, adjusting solvents, adjusting pH, ultrasonic field forces, electro-osmotic field forces, iontophoretic field forces, electrophoretic field forces and combinations thereof. The duration of the osmotic force may be manipulated using electric field forces, magnetic field forces, electromagnetic field forces, biochemical reactions, chemicals, molarity adjustment, adjusting solvents, adjusting pH, ultrasonic field forces, electrosmotic field forces, iontophoretic field forces, electrophoretic field forces and combinations thereof. The receiver may combine the osmotic agent and the biochemical reagents to detect the presence of the analyte. The reagents may allow the use of electrochemical, biochemical, optical, fluorescence, absorbance, reflectance, Raman, magnetic, mass spectrometry, IR spectroscopy measurement methods and combinations thereof to be performed on the receiver. The receiver may also be removed periodically for detection.

In one embodiment, the intensity, duration, and frequency of exposure of biological membrane to osmotic forces may be manipulated by using an electric current to cause a change in the concentration of the osmotic agent that is in contact with the ultrasound-exposed biological membrane. The osmotic agent may be a multi-charged agent that can dissociate into several charged species. These charged species may be transported using electric field forces. A membrane may be used to isolate the charged species. The charged species freely diffuse and combine upon removal of the electric field force.

In one embodiment, the intensity, duration, and frequency of exposure of biological membrane to osmotic forces may be manipulated by using active forces to cause a change in the concentration of the osmotic agent that is in contact with the ultrasound-exposed biological membrane. The osmotic agent may be a neutral charge agent. The agent may be transported using a variety of field forces. The field force depends on the constitutive and colligative properties of the chosen agent. The field force generates a force necessary to move the osmotic agent toward and away from the biological membrane surface. The movement of the osmotic agent modulates the periodic and continuous extraction of body fluid through the stratum corneum.

In one embodiment, the intensity, duration, and frequency of exposure of biological membrane to osmotic forces may be manipulated by changing the concentration of the osmotic agent that is in contact with the ultrasound-exposed biological membrane. Manipulating the volume of the solvent and the volume of the hydrogel containing the osmotic agent may cause a change in the concentration of the osmotic agent. The volume of the hydrogel can be changed by constructing a hydrogel wherein its volume is sensitive to the concentrations of molecules that can diffuse into the gel. One example is a hydrogel constructed to be sensitive to the molecule glucose. The hydrogel volume can also be changed by manipulating its temperature and by changing the pH of the gel.

According to another embodiment of the present invention, a drug delivery patch apparatus is disclosed. The apparatus includes an ultrasound transducer for applying ultrasound to a membrane. The membrane may include biological membranes, synthetic membranes, or a cell culture. A biological membrane may include skin, mucosal and buccal membranes. The apparatus further includes a power source coupled to the transducer. The apparatus further includes drug molecules between the transducer and the membrane, and an attaching device that attaches the apparatus to the membrane. According to another embodiment, the apparatus further includes drive electronics coupled to the transducer such that the drive electronics enables the transducer to apply ultrasound. According to another embodiment, the apparatus further includes an interface coupled to the drive electronics.

According to another embodiment of the present invention, a method for transdermal vaccination by sonophoresis is disclosed. According to the one embodiment, the method comprises the steps of enhancing the permeability of the skin by the application of ultrasound; providing a vaccine to the permeabilized skin, and delivering the vaccine to the skin cells, for example, Langerhans cells, dendritic cells, and keratinocytes. The steps of increasing the permeability of the skin and providing a vaccine to the permeabilized skin may occur simultaneously.

In another embodiment of the present invention, ultrasound is used to irritate or inflame an area of skin. Next, a vaccine is provided to the irritated or inflamed skin. This is more effective in inducing the immune response of the body.

In another embodiment of the present invention, ultrasound is used to deliver an immunomodulatory agent such as adjuvant to the skin to induce cells such as macrophages or monocytes to migrate to the site. Next, the vaccine is delivered to the site. This enhances the immune response specific to the antigen delivered. The mode of adjuvant delivery can vary. For example, the adjuvant be included in the bellows cartridge of the ultrasound device and delivered to the skin during sonopermeation. Conversely, the immunomodulatory agent can be applied to the skin after ultrasound permeation.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 is a flowchart depicting a method for non-invasive body fluid sampling according to one embodiment of the present invention;

FIG. 2 depicts a device for controlled application of ultrasound to a biological membrane to enhance the permeability of the biological membrane according to one embodiment of the present invention;

FIG. 3 depicts the components to perform discrete extraction and measurement of body fluid to infer analyte concentrations according to one embodiment of the present invention;

FIG. 4 depicts the components to perform continuous extraction and measurement of body fluid to infer analyte concentrations according to one embodiment of the present invention;

FIG. 5 depicts an approach to periodic monitoring of an analyte by performing periodic osmotic extractions of body fluid according to one embodiment of the present invention;

FIG. 6 depicts the components of a wearable extraction chamber according to one embodiment of the present invention;

FIG. 7 depicts a graph of glucose flux versus blood glucose concentration according to one embodiment of the present invention;

FIG. 8 depicts a flow chart of a method for controlled enhancement of transdermal delivery according to one embodiment of the present invention;

FIG. 9 depicts an apparatus for performing continuous transdermal analyte monitoring according to one embodiment of the present invention;

FIG. 10 is a drawing of the sensor body shown in FIG. 9 from a first view;

FIG. 11 is a drawing of the apparatus shown in FIG. 9 from a second view;

FIG. 12 illustrates a drug delivery patch apparatus in accordance with one embodiment of the present invention;

FIG. 13 illustrates a cross-sectional view of a transducer in accordance with one embodiment of the present invention;

FIG. 14 illustrates a drug delivery patch apparatus having a feedback mechanism in accordance with one embodiment of the present invention;

FIG. 15 depicts a flowchart of the method for transdermal vaccination by sonophoresis according to one embodiment of the present invention;

FIG. 16a is a graph of a pain/discomfort score plotted as a function of skin pretreatment agent according to an exemplary embodiment of the invention;

FIG. 16b is a graph of the success rate of sonication plotted as a function of skin treatment agent according to an exemplary embodiment of the invention;

FIG. 17a is a graph of skin impedance obtained on ultrasonicated skin (dorsum, anticubital) as a function of skin pretreatment according to an exemplary embodiment of the invention;

FIG. 17b is a graph of percent success rate of sonication obtained in human volunteers according to an exemplary embodiment of the invention;

FIG. 17c is a graph of average pain/discomfort score associated with ultrosonication in human volunteers according to an exemplary embodiment of the invention;

FIG. 17d is a graph of the time required to achieve a successful sonication in human volunteers according to an exemplary embodiment of the invention;

FIG. 18a is a graph of success rate of sonication obtained in human volunteers as a function of skin treatment according to an exemplary embodiment of the invention;

FIG. 18b is a graph of average pain/discomfort score associated with ultrasonication in human volunteers according to an exemplary embodiment of the invention;

FIG. 19a is a graph of success rate of sonication obtained in human volunteers as a function of various skin treatment methods on the volar forearm according to an exemplary embodiment of the invention;

FIG. 19b is a graph of average pain/discomfort score associated with ultrasonication in human volunteers according to an exemplary embodiment of the invention;

FIG. 20 is a graph of sensor response versus blood glucose levels according to an exemplary embodiment of the invention; and

FIG. 21 is a graph of sensor response versus blood glucose levels according to another exemplary embodiment of the invention.

### DETAILED DESCRIPTION OF THE INVENTION

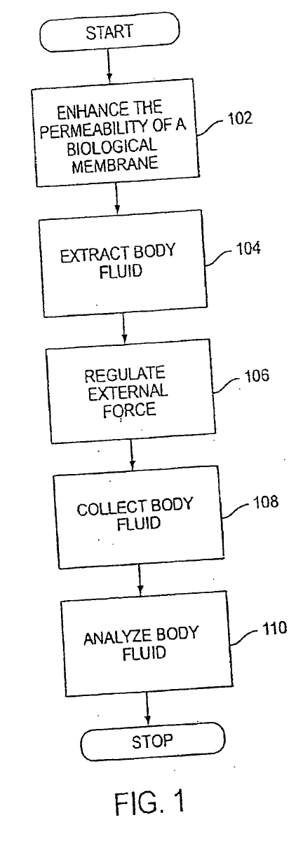

The preferred embodiments of the present invention and their advantages are best understood by referring to FIGS. 1 through 21 of the drawings, like numerals being used for like and corresponding parts of the various drawings.

According to exemplary embodiments of the invention, a hydration process can be applied to a biological membrane before and/or during and/or after sonication to enhance transdermal transport. The method can be applied to enhance transdermal extraction of analytes, transdermal drug delivery, and/or transdermal vaccination, for example.

As used herein, the term “body fluid” may include blood, interstitial fluid, lymph, and/or analyte. In addition, as used herein, the term “biological membrane” may include tissue, mucous membranes and comified tissues, including skin, buccal, and nails. Further, as used herein, the term “force” may also include force gradients.

Although the present invention may be described in conjunction with human applications, veterinary applications are within the contemplation and the scope of the present invention.

Referring to FIG. 1, a flowchart depicting a method for non-invasive body fluid sampling and analysis according to one embodiment of the present invention is provided. In step 102, the permeability of an area of biological membrane is enhanced. In one embodiment, the area of biological membrane may be located on the volar forearm of a mammalian subject. In another embodiment, the area of biological membrane may be located on a thigh of a mammalian subject. In yet another embodiment, the area of biological membrane may be located on the abdomen. In still another embodiment, the area of biological membrane may be located on the back. Other body locations may also be used.

In general, several techniques may be used to enhance the permeability of the biological membrane, such as creating physical micropores, physically disrupting the lipid bilayers, chemically modifying the lipid bilayers, physically disrupting the stratum corneum, and chemically modifying the stratum corneum. The creation of micropores, or the disruption thereof, may be achieved by physical penetration using a needle, a microneedle, a silicon microneedle, a laser, a laser in combination with an absorbing dye, a heat source, an ultrasonic needle, an ultrasonic transducer, cryogenic ablation, RF ablation, photo-acoustic ablation, and combinations thereof.

In a preferred embodiment, ultrasound may be applied to the area of biological membrane to enhance its permeability. Ultrasound is generally defined as sound at a frequency of greater than about 20 kHz. Therapeutic ultrasound is typically between 20 kHz and 5 MHz. Near ultrasound is typically about 10 kHz to about 20 kHz. It should be understood that in addition to ultrasound, near ultrasound may be used in embodiments of the present invention.

In general, ultrasound, or near ultrasound, is preferably applied to the area of biological membrane at a frequency sufficient to cause cavitation and increase the permeability of the biological membrane. In one embodiment, ultrasound may be applied at a frequency of from about 10 kHz to about 500 kHz. In another embodiment, ultrasound may be applied at a frequency of from about 20 kHz to about 150 kHz. In yet another embodiment, the ultrasound may be applied at 50 kHz. Other frequencies of ultrasound may be applied to enhance the permeability level of the biological membrane.

In one embodiment, the ultrasound may have an intensity in the range of about 0 to about 100 watt/cm², and preferably in the range of 0 to about 20 watt/cm². Other appropriate intensities may be used as desired.

Techniques for increasing the permeability of a biological membrane are disclosed in U.S. Pat. No. 6,190,315 to Kost et al., the disclosure of which is hereby incorporated by reference in its entirety.

In step 104, body fluid is extracted through or out of the area of biological membrane. In one embodiment, an external force, such as an osmotic force, may assist in the extraction. In one embodiment, the osmotic force may be controlled before, during, and after the permeability of the biological membrane is enhanced.

In one embodiment, the osmotic force may be generated by the application of an osmotic agent to the area of biological membrane. The osmotic agent may be in the form of an element, a molecule, a macromolecule, a chemical compound, or combinations thereof. The osmotic agent may also be combined with a liquid solution, a hydrogel, a gel, or an agent having a similar function.

In step 106, the magnitude, intensity, and duration of the external force may be regulated by at least one additional first energy and/or force. In one embodiment, the first additional energy and/or force may be applied to control and regulate the movement and function of the osmotic agent for extraction of body fluid through and out of the biological membrane. The first additional energy and/or force may be provided in the form of heat, a temperature force, a pressure force, an electromotive force, a mechanical agitation, ultrasound, iontophoresis, an electromagnetic force, a magnetic force, a photothermal force, a photoacoustic force, and combinations thereof. The effect of an electric field and ultrasound on transdermal drug delivery is disclosed in U.S. Pat. No. 6,041,253, the disclosure of which is incorporated, by reference, in its entirety.

In one embodiment, if the first additional energy and/or force is provided by ultrasound, the frequency of the ultrasound may be provided at a different frequency than the frequency used to enhance the permeability of the biological membrane. In one embodiment, the frequency of the first additional energy/force ultrasound may be higher than the frequency of the permeability enhancing ultrasound.

In step 108, the body fluid may be collected in a receiver. In one embodiment, the receiver may be contacted with the biological membrane in a form of a patch, a wearable reservoir, a membrane, an absorbent strip, a hydrogel, or a structure that performs an equivalent function. Other types and configurations of receivers may be used.

In one embodiment, the receiver may be provided with a secondary receiver having an analyte concentration that is continuously maintained to be substantially lower than the analyte concentration in the body fluid, so the chemical concentration driving force between body fluid and secondary receiver is maximized. This may be achieved by chemical reaction or volume for dilution or similar means.

In one embodiment, a second external energy/force may be applied between the first receiver and the secondary receiver. In one embodiment, the second external energy/force may be different (e.g., a different type of external force) from the first external energy/force. In another embodiment, the second external energy/force may be the same (e.g., the same type of external force) as the first external energy/force. The first and second external energy/force may vary in type, duration, and intensity, and may be controlled through different additional energy and/or forces.

In step 10, the collected body fluid may be analyzed. In one embodiment, the analysis may include the use of appropriate methods, such as electrochemical, biochemical, optical, fluorescence, absorbance, reflectance, Raman, magnetic, mass spectrometry, infra-red (IR) spectroscopy measurement, and combinations thereof.

In one embodiment, multiple analytes may be analyzed simultaneously, in parallel, or in series. The results from these multiple analyses may be used in combination with algorithms, for example, to increase the accuracy, or precision, or both, of the analysis and measurements.

In one embodiment, the receiver may be removed from contact with the biological membrane in order to analyze the collected body fluid. In another embodiment, the receiver may remain in contact with the biological membrane as the collected body fluid is analyzed.

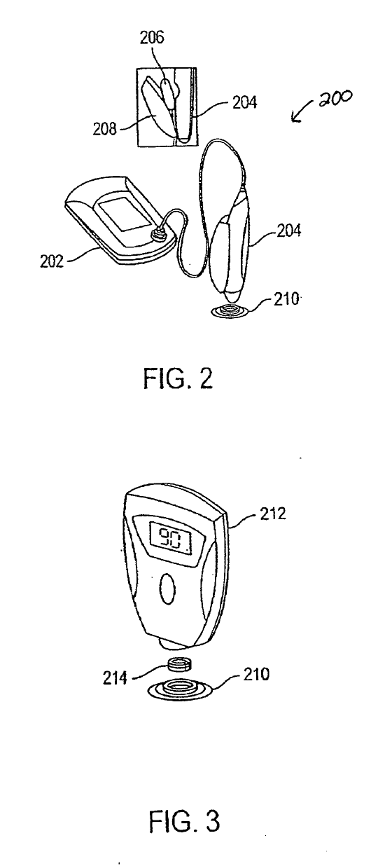

Referring to FIG. 2, a device for the controlled application of ultrasound to biological membrane to enhance the permeability of a biological membrane according to one embodiment of the present invention is shown. Device 200 includes controller 202, which interfaces with ultrasound applicator 204 by any suitable means, such as a cable. Controller 202 controls the application of ultrasound to the area of biological membrane. In one embodiment, ultrasound or near ultrasound having an intensity in the range of about 0 to about 20 watt/cm² may be generated by controller 202 and ultrasound applicator 204. In one embodiment, the ultrasound may have a frequency of about 20 kHz to about 150 kHz. In another embodiment, the ultrasound may have a frequency of 50 kHz. Other ultrasound frequencies may also be used.

In addition, controller 202 may include a display, such as a LCD or a LED display, in order to convey information to the user as required. Controller 202 may also include a user interface as is known in the art.

Ultrasound applicator 204 may be provided with cartridge 206, which contains ultrasound coupling solution 208. Cartridge 206 may be made of any material, such as plastic, that may encapsulate ultrasound coupling solution 208. Suitable ultrasound coupling solutions 208 include, but are not limited to, water, saline, alcohols including ethanol and isopropanol (in a concentration range of 10 to 100% in aqueous solution), surfactants such as Triton X-100, SLS, or SDS (preferably in a concentration range of between 0.001 and 10% in aqueous solution), DMSO (preferably in a concentration range of between 10 and 100% in aqueous solution), fatty acids such as linoleic acid (preferably in a concentration range of between 0.1 and 2% in ethanol-water (50:50) mixture), azone (preferably in a concentration range of between 0.1 and 10% in ethanol-water (50:50) mixture), polyethylene glycol in a concentration range of preferably between 0.1 and 50% in aqueous solution, histamine in a concentration range of preferably between 0.1 and 100 mg/ml in aqueous solution, EDTA in a concentration range of preferably between one and 100 mM, sodium hydroxide in a concentration range of preferably between one and 100 mM, sodium octyl sulfate, N-tauroylsarcosine, octyltrimethyl ammoniumbromide, dodecyltrimethyl ammoniumbromide, tetradecyltrimethyl ammoniumbromide, hexadecyltrimethyl ammoniumbromide, dodecylpyridinium chloride hydrate, SPAN 20, BRIJ 30, glycolic acid ethoxylate 4-ter-butyl phenyl ether, IGEPAL CO-210, and combinations thereof.

In one embodiment, the coupling medium may also include a chemical enhancer. Transport enhancement may be obtained by adding capillary permeability enhancers, for example, histamine, to the coupling medium. The concentration of histamine in the coupling medium may be in the range of between 0.1 and 100 mg/ml. These agents may be delivered across the biological membrane during application of ultrasound and may cause local edema that increases local fluid pressure and may enhance transport of analytes across the biological membrane. In addition, the occurrence of free fluid due to edema may induce cavitation locally so as to enhance transport of analytes across the biological membrane.

In one embodiment, cartridge 206 may be pierced when inserted into ultrasound applicator 204, and ultrasound coupling solution 208 may be transferred to a chamber (not shown).

A target-identifying device, such as target ring 210, may be attached to the area of biological membrane that will have its permeability increased. Target ring 210 may be attached to the area of biological membrane by a transdermal adhesive (not shown). In one embodiment, target ring 210 may have the transdermal adhesive pre-applied, and may be disposed after each use. In another embodiment, target ring 210 may be reusable.

Target ring 210 may be made of any suitable material, including plastic, ceramic, rubber, foam, etc. In general, target ring 210 identifies the area of biological membrane for permeability enhancement and body fluid extraction. In one embodiment, target ring 210 may be used to hold receiver 214 in contact with the biological membrane after the permeability of the biological membrane has been increased.

In one embodiment, target ring 210 may be used to monitor the permeability level of the biological membrane, as disclosed in PCT International Patent Appl'n Ser. No. PCT/US99/30067, entitled “Method and Apparatus for Enhancement of Transdermal Transport,” the disclosure of which is incorporated by reference in its entirety. In such an embodiment, target ring 210 may interface with ultrasound applicator 204.

Ultrasound applicator 204 may be applied to target ring 210 and activated to expose ultrasound coupling solution 208 to the biological membrane. Controller 202 controls ultrasound applicator 204 to transmit ultrasound through ultrasound coupling solution 208. During ultrasound exposure, controller 202 may monitor changes in biological membrane permeability, and may display this information to the user.

Controller 202 may cease, or discontinue, the application of ultrasound once a predetermined level of biological membrane permeability is reached. This level of permeability may be preprogrammed, or it may be determined in real-time as the ultrasound is applied. The predetermined level of permeability may be programmed for each individual due to biological membrane differences among individuals.

After the predetermined level of permeability is reached, ultrasound coupling solution 208 may be vacuated from chamber (not shown) into cartridge 206, which may then be discarded. In another embodiment, ultrasound coupling solution 208 may be vacuated into a holding area (not shown) in ultrasound applicator 204, and later discharged. Ultrasound applicator 204 may then be removed from target ring 210.

Referring to FIG. 3, a device for the analysis of body fluid according to one embodiment of the present invention is provided. Receiver 214 may be placed into target ring 210 to perform a discrete, or on-demand, extraction of body fluid through and/or out of the biological membrane. Receiver 214 may contain a medium, such as a hydrogel layer, that incorporates an osmotic agent. In one embodiment, the hydrogel may be formulated to contain phosphate buffered saline (“PBS”), with the saline being sodium chloride having a concentration range of about 0.01 M to about 10 M. The hydrogel may be buffered at pH 7. Other osmotic agents may also be used in place of, or in addition to, sodium chloride. Preferably, these osmotic agents are non-irritating, non-staining, and non-immunogenic. Examples of such osmotic agents include, inter alia, lactate and magnesium sulfate.

In another embodiment, receiver 214 may include a fluid or liquid medium, such as water or a buffer that is contained within a semi-permeable membrane. Receiver 214 may also include a spongy material, such as foam.

Receiver 214 may be applied to the biological membrane to contact the ultrasound exposed biological membrane. In one embodiment, receiver 214 may be applied to the biological membrane for a time period sufficient to collect an amount of body fluid sufficient for detection. In another embodiment, receiver 214 may be applied to the biological membrane for a sufficient time period to collect a predetermined amount of body fluid. In yet another embodiment, receiver 214 may be applied to the biological membrane for a predetermined time. In one embodiment, the contact between receiver 214 and the biological membrane may last for 15 minutes or less. In another embodiment, the contact between receiver 214 and the biological membrane may last for 5 minutes or less. In still another embodiment, the contact between receiver 214 and the biological membrane may last for 2 minutes or less. The actual duration of contact may depend on the sensitivity of the detection method used for analysis.

In one embodiment, the medium of receiver 214 may contain at least one reagent (not shown) in order to detect the presence of certain analytes in the body fluid that has been extracted from or through the biological membrane. In one embodiment, the hydrogel layer of receiver 214 may contain the reagents, and the reagents may be attached to the hydrogel by ionic and/or covalent means, or may be immobilized by gel entrapment. The reagents may also be arranged as an adjacent layer to the hydrogel wherein the analyte from the body fluid that has been extracted into the hydrogel can diffuse into and react to generate by-products. The by-products may then be detected using electrochemical, biochemical, optical, fluorescence, absorbance, reflectance, Raman, magnetic, mass spectrometry, IR spectroscopy measurement methods and combinations thereof.

The detection methods may be performed by meter 212. Meter 212 may include a processor (not shown) and a display, such as an LCD display. Other suitable displays may be provided.

In one embodiment, meter 212 may provide an interface that allows information be downloaded to an external device, such as a computer. Such an interface may allow the connection of interface cables, or it may be a wireless interface.

Meter 212 may be configured to determine body fluid glucose concentration by incorporating glucose oxidase in the medium of receiver 214. In one embodiment, glucose from extracted body fluid may react with glucose oxidase to generate hydrogen peroxide. Hydrogen peroxide may be detected by the oxidation of hydrogen peroxide at the surface of electrodes incorporated into receiver 214. The oxidation of hydrogen peroxide transfers electrons onto the electrode surface, which generates a current flow that can be quantified using a potentiostat, which may be incorporated into meter 212. A glucose concentration proportional to the concentration of hydrogen peroxide may be calculated, and the result may be reported to the user via a display. Various configurations of electrodes and reagents, known to those of ordinary skill in the art, may be incorporated to perform detection and analysis of glucose and other analytes.

Meter 212 may also be configured to simultaneously measure the concentration of an analyte, such as glucose, where the body fluid concentration is expected to fluctuate, and an analyte, like creatinine or calcium, where the body fluid concentration is expected to remain relatively stable over minutes, hours, or days. An analyte concentration, which may be determined by an algorithm that takes into account the relative concentrations of the fluctuating and the more stable analyte, may be reported to the user via a display.

In another embodiment, meter 212 may analyze multiple analytes simultaneously, in parallel, or in series. The results from these multiple analyses may be used in combination with algorithms, for example, to increase the accuracy, or precision, or both, of the analysis and measurements.

Receiver 214 may be discarded after the extraction and measurement steps. In another embodiment, receiver 214 may be reused. In one embodiment, receiver 214 may be cleaned, sanitized, etc. before it may be reused. Various configurations of electrodes and reagents, known to those of ordinary skill in the art, may be incorporated to perform detection and analysis of glucose and other analytes.

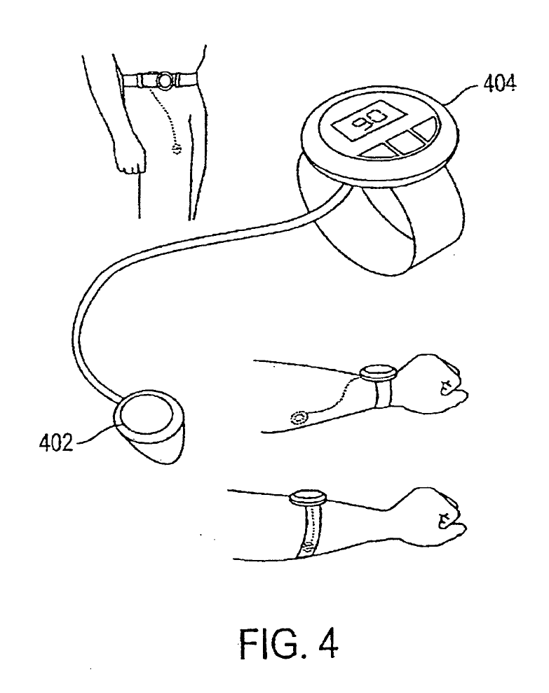

Referring to FIG. 4, a device for the continuous extraction and analysis of body fluid to infer analyte concentrations according to another embodiment of the present invention is provided. As shown in the figure, a biological membrane site on the forearm, the abdomen, or thigh may be exposed to ultrasound; other biological membrane sites, such as those on the back, may also be used. Receiver 402, which may be similar to receiver 214, may contact the ultrasound exposed biological membrane site to perform continuous extraction of body fluid. In one embodiment, receiver 402 may contain a medium, such as a hydrogel layer, that may incorporate an osmotic agent, such as sodium chloride. The hydrogel is formulated to contain phosphate buffered saline (PBS), with the saline being sodium chloride in the concentration range of 0.01 M to 10 M. The hydrogel may be buffered at pH 7.

Other osmotic agents may also be used in place of, or in addition to, sodium chloride. These osmotic agents are preferably non-irritating, non-staining, and non-immunogenic. Examples of these other osmotic agents may include, inter alia, lactate and magnesium sulfate. Receiver 402 may be applied to contact the ultrasound exposed biological membrane. In one embodiment, the duration of this contact may be 12-24 hours, or more. In another embodiment, other durations of contact, including substantially shorter durations, and substantially longer durations, may be used as desired.

In another embodiment, receiver 402 may include a fluid or liquid medium, such as water or a buffer that is contained within a semi-permeable membrane. Receiver 402 may also include a spongy material, such as foam.

In one embodiment, the medium of receiver 402 may contain at least one reagent (not shown) that detects the presence of analytes in the body fluid that has been extracted thorough and out of the biological membrane. In one embodiment, the hydrogel layer of receiver 402 may contain reagents that may be attached by ionic and covalent means to the hydrogel, or may be immobilized by gel entrapment. The reagents may also be arranged as an adjacent layer to the hydrogel wherein the analyte from the body fluid that has been extracted into the hydrogel may diffuse into and react to generate by-products. The by-products may be detected using electrochemical, biochemical, optical, fluorescence, absorbance, reflectance, Raman, magnetic, mass spectrometry, IR spectroscopy measurement methods and combinations thereof.

The detection methods and results may be performed and presented to the user by meter 404, which may be similar in function to meter 212, discussed above. In one embodiment, meter 404 may be wearable. For example, as depicted in the figure, meter 404 may be worn in a manner similar to the way a wristwatch is worn. Meter 404 may also be worn on a belt, in a pocket, etc.

Meter 404 may incorporate power and electronics to control the periodic extraction of body fluid, to detect analyte, and to present the analyte concentration in a continuous manner. Meter 404 may contain electronics and software for the acquisition of sensor signals, and may perform signal processing, and may store analysis and trending information.

In one embodiment, meter 404 may provide an interface that allows information be downloaded to an external device, such as a computer. Such an interface may allow the connection of interface cables, or it may be a wireless interface.

Meter 404 may be configured to determine body fluid glucose concentration by incorporating glucose oxidase in the medium. In one embodiment, glucose from extracted body fluid may react with glucose oxidase to generate hydrogen peroxide. Hydrogen peroxide may be detected by the oxidation of hydrogen peroxide at the surface of electrodes incorporated into receiver 402. The oxidation of hydrogen peroxide transfers electrons onto the electrode surface, which generates a current flow that can be quantified using a potentiostat, which may be incorporated into meter 404. A glucose concentration proportional to the concentration of hydrogen peroxide may be calculated and the result may be reported to the user via a display. Various configurations of electrodes and reagents, known to those of ordinary skill in the art, may be incorporated to perform detection and analysis of glucose and other analytes.

In one embodiment, meter 404 may also be configured to simultaneously measure concentration of an analyte, such as glucose, where the body fluid concentration is expected to fluctuate, and an analyte, like creatinine or calcium, where the body fluid concentration is expected to remain relatively stable over minutes, hours, or days. An analyte concentration, which may be determined by an algorithm that takes into account the relative concentrations of the fluctuating and the more stable analyte, may be reported to the user via a display.

In another embodiment, meter 404 may analyze multiple analytes simultaneously, in parallel, or in series. The results from these multiple analyses may be used in combination with algorithms, for example, to increase the accuracy, or precision, or both, of the analysis and measurements.

In another embodiment, receiver 402 may be removed from contact with the biological membrane for analysis by meter 404. Receiver 402 may be put in contact with the biological membrane after such analysis.

Meter 404 may provide analyte readings to the user in a periodic or a continuous manner. For example, in one embodiment, in continuous monitoring of the analyte glucose, glucose concentration may be displayed to the user every 30 minutes, more preferably every 15 minutes, most preferable every 5 minutes, or even more frequently. In another embodiment, the glucose concentration may be displayed continuously. The period may depend on the sensitivity and method of analyte detection. In continuous glucose monitoring, in one embodiment, glucose detection may be performed by an electrochemical method using electrodes and reagents incorporated into receiver 402 and detection and analysis performed by meter 404. During the measurement period, osmotic extraction of body fluid may be performed continuously by the hydrogel layer of receiver 402. Body fluid may accumulate in the hydrogel of receiver 402. Glucose in body fluid diffuses to react with glucose oxidase and is converted into hydrogen peroxide. The hydrogen peroxide is consumed by poising the working electrode with respect to a reference electrode. During the resting period, hydrogen peroxide accumulates and is consumed or destroyed before the measuring period. The magnitude of the working potential can be applied to rapidly consume the build up of hydrogen peroxide.

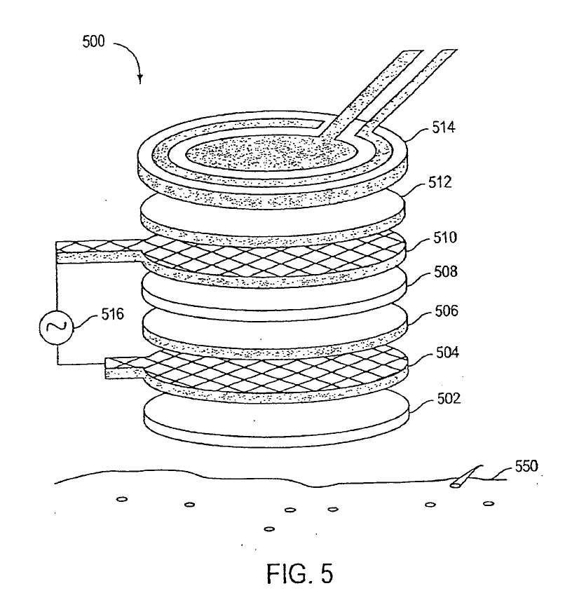

Referring to FIG. 5, an approach to periodic monitoring of an analyte by performing periodic osmotic extractions of body fluid according to another embodiment of the present invention is shown. The osmotic extraction intensity and frequency may be manipulated by using an osmotic agent that dissociates into multiple charged species, and an electrical potential may be used to move the concentration of charges toward and away from biological membrane surface 550. Receiver 500 may include grid, mesh, or screen 504; medium 506, which may be a hydrogel layer; membrane 508; counter grid, mesh, or screen 510; oxidase layer 512; and detection layer 514. Grid 504 and counter grid 510 may be connected to voltage source 516. Membrane 508 may be a semi-permeable membrane that is used to induce a concentration gradient barrier for the osmotic agent contained in medium 506. The preferable osmotic agent may contain negative and positive species or counter ions. Manipulating the concentration of charged species at the boundary adjacent to the stratum corneum of the ultrasound-exposed biological membrane may provide periodic extraction of body fluid.

In one embodiment, receiver 500 may make contact with the skin though contact medium 502, which may be a hydrogel, or other suitable medium.

The concentration of the charged species may be manipulated by applying a potential difference between grid 504 and counter grid 510 using voltage source 516. In one embodiment, the potential difference may be of a magnitude that is sufficient to manipulate the osmotic agent. The polarity of the grid may also be changed to transport charges toward and away from biological membrane surface 550. Grid 504 and counter grid 510 may be configured with optimum porosity as to allow body fluid and/or analyte to travel out of stratum corneum, through grid 504, through grid 510, and into oxidase layer 512, and ultimately to detection layer 514. Oxidase layer 512 may be used with an appropriate catalyst, or enzyme, to confer specificity of analyte detection. Detection layer 514 may include working and reference electrodes (not shown) that allow for the detection of the by-products of oxidase layer 512 to quantify the concentration of the desired analyte of detection.

### EXAMPLE 1

The following example does not limit the present invention in any way, and is intended to illustrate an embodiment of the present invention.

The following is a description of experiments which implemented painless extraction, collection, and analysis of body fluid to determine body fluid glucose concentration in a human using a hyperosmotic extraction fluid and comparing this condition with iso-osmotic extraction fluid, in accordance with one embodiment of the present invention. Although body fluid glucose concentration serves as an example to demonstrate feasibility, other analytes are within the contemplation of the present invention. In addition, multiple analytes may be measured and/or analyzed simultaneously, in parallel, or in series, and results from these multiple measurements may be used in combination with algorithms, for example, to increase the accuracy or precision or both of measurements. As may be recognized by one of ordinary skill in the art, these steps may be automated and implemented with the device described above.

Four sites on the volar forearm of a human volunteer were treated with ultrasound using the device described in FIG. 2. The ultrasound transducer and its housing were placed on the volar forearm of the volunteer with enough pressure to produce a good contact between the skin and the outer transducer housing, and to prevent leaking. The area surrounding the transducer was then filled with a coupling medium of sodium dodecyl sulfate and silica particles in phosphate-buffered saline (PBS). Ultrasound was briefly applied (5-30 s), the transducer apparatus was removed from the biological membrane, and the skin was rinsed with tap water and dried.

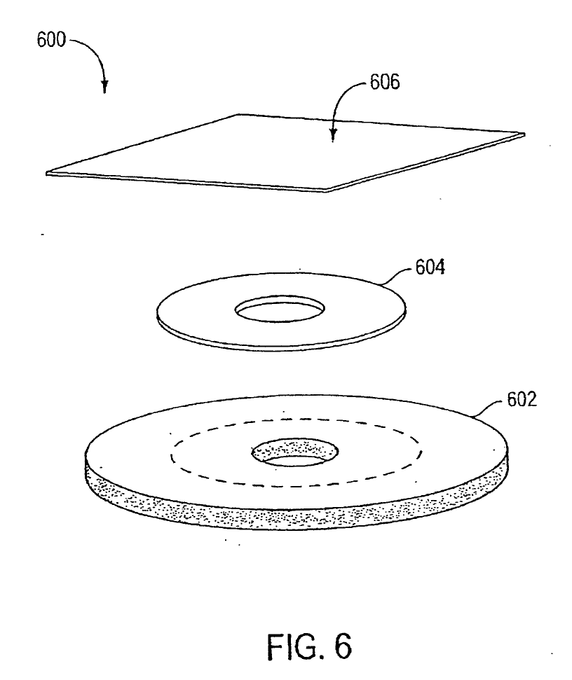

FIG. 6 describes the components of wearable extraction chamber 600. Four extraction chambers were placed on each sonicated site of the human volunteer. Thin circular foam chamber 602 was constructed using foam MED 5636 Avery Dennison ( 7/16″ ID× 11/8″ OD). Foam chambers 602 were attached concentrically to the sonicated biological membrane sites using double-sided adhesive (Adhesive Arcade 8570, 7/16″ ID×⅞″ OD) attached to one side of element 602. The other side of foam chamber 602 was attached concentrically to double-sided adhesive 604 (Adhesive Arcade 8570, 7/16″ ID×⅞″ OD). Thin transparent lid 606 was made of 3M Polyester 1012 ( 11/8″× 11/8″). Double-sided adhesive 604 permitted thin transparent lid 606 to be attached to foam chamber 602 after placement of liquid into the inner diameter of foam chamber 602 when attached to biological membrane. Thin transparent lid 606 acted as a lid to prevent liquid from leaking out of the extraction chamber, and to allow the extraction chambers to be wearable for an extended period of time.

Each extraction chamber was alternately filled with 100 μL of extraction solution for 15 min and 100 μL hydration solution for 10-40 min. Extraction solution was PBS. On two sites the PBS contained additional NaCl to bring the total concentration of NaCl to 1 M. Hydration solution was PBS for all sites.

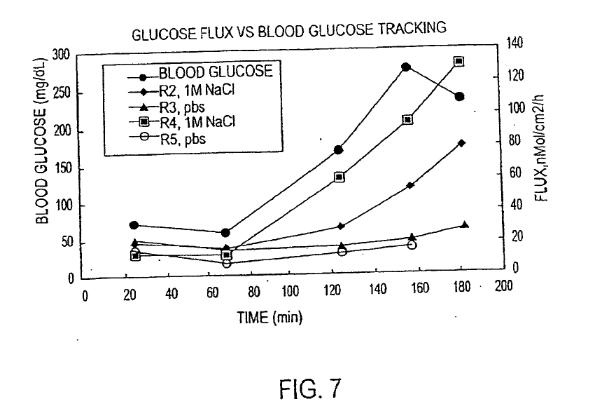

Solutions were collected and analyzed for glucose concentration using high-pressure liquid chromatography. The results of the HPLC concentration were normalized for the injection amount and the total solution volume, and were reported as glucose flux (Q_{g}), the mass of glucose that crossed the sonicated site per unit time per unit area. Body fluid glucose concentrations (C_{bg}) were obtained by testing capillary blood obtained from a lanced finger in a Bayer Glucometer Elite meter. It was hypothesized that Q_{g} would be linearly proportional to C_{bg}. FIG. 7 shows a graph of Q_{g} versus C_{bg}. Unexpectedly, Q_{g} from the sonicated sites exposed to 1 M NaCl correlated to C_{bg} much more strongly than Q_{g} from the sonicated sites exposed to 0.15 M NaCl.

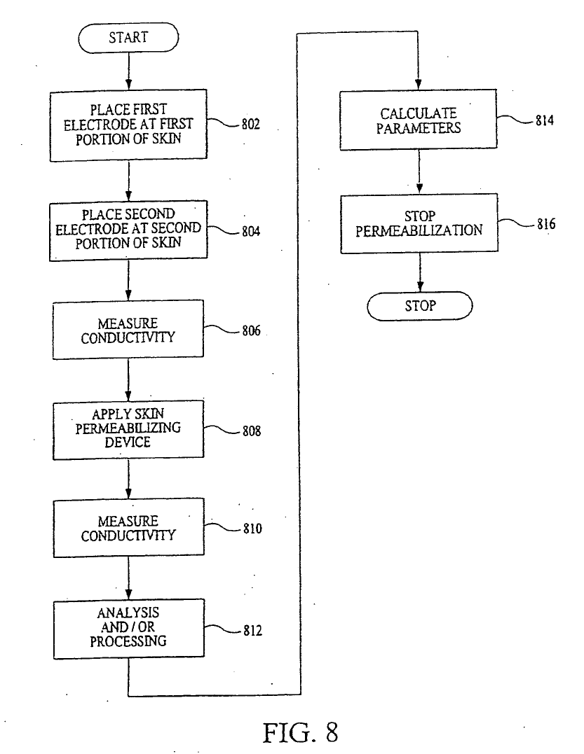

According to another aspect of the present invention, an apparatus and method for regulating the degree of skin permeabilization through a feedback system is provided. This apparatus and method may be similar to what has been described above, with the addition of further regulation of the degree of skin permeabilization. Feedback control as a method of monitoring the degree of skin permeability is described in more detail in U.S. application Ser. No. 09/868,442, which is incorporated by reference in its entirety. In this embodiment, the application of the skin-permeabilizing device is terminated when desired values of parameters describing skin conductance are achieved. As the discussion proceeds with regard to FIG. 8, it should be noted that the descriptions above may be relevant to this description.

Referring to FIG. 8, a flowchart of the method is provided. In step 802, a first, or source, electrode is coupled in electrical contact with a first area of skin where permeabilization is required. The source electrode does not have to make direct contact with the skin. Rather, it may be electrically coupled to the skin through the medium that is being used to transmit ultrasound. In one embodiment, where an ultrasound-producing device is used as the skin permeabilizing device, the ultrasonic transducer and horn that will be used to apply the ultrasound doubles as the source electrode through which electrical parameters of the first area of skin may be measured and is coupled to the skin through a saline solution used as an ultrasound medium. In another embodiment, a separate electrode is affixed to the first area of skin and is used as the source electrode. In still another embodiment, the housing of the device used to apply ultrasound to the first area of skin is used as the source electrode. The source electrode can be made of any suitable conducting material including, for example, metals and conducting polymers.

Next, in step 804, a second, or counter, electrode is coupled in electrical contact with a second area of skin at another chosen location. This second area of skin can be adjacent to the first area of skin, or it can be distant from the first area of skin. The counter electrode can be made of any suitable conducting material including, for example, metals and conducting polymers.

When the two electrodes are properly positioned, in step 806, an initial conductivity between the two electrodes is measured. This may be accomplished by applying an electrical signal to the patch of skin through the electrodes. In one embodiment, the electrical signal supplied may have sufficient intensity so that the electrical parameter of the skin can be measured, but have a suitably low intensity so that the electrical signal does not cause permanent damage to the skin, or any significant electrophoresis effect for the substance being delivered. In one embodiment, a 10 Hz AC source is used to create a voltage differential between the source electrode and the counter electrode. The voltage supplied should not exceed 500 mV, and preferably not exceed 100 mV, or there will be a risk of damaging the skin. In another embodiment, an AC current source is used. The current source may also be suitably limited. The initial conductivity measurement is made after the source has been applied using appropriate circuitry. In one embodiment, a resistive sensor is used to measure the impedance of the patch of skin at 10 Hz. In another embodiment, a 1 kHz source is used. Sources of other frequencies are also possible.

In step 808, a skin-permeabilizing device is applied to the skin at the first site. Any suitable device that increases the permeability of the skin may be used. In one embodiment, ultrasound is applied to the skin at the first site. According to one embodiment, ultrasound having a frequency of 20 kHz and an intensity of about 10 W/cm² is used to enhance the permeability of the patch of skin to be used for transdermal transport.

In step 810, the conductivity between the two sites is measured. The conductivity may be measured periodically, or it may be measured continuously. The monitoring measurements are made using the same electrode set up that was used to make the initial conductivity measurement.

In step 812, mathematical analysis and/or signal processing may be performed on the time-variance of skin conductance data. Experiments were performed on human volunteers according to the procedure above, with ultrasound used as the method of permeabilization. Ultrasound was applied until the subjects reported pain. Skin conductivity was measured once every second during ultrasound exposure. After plotting the conductance data, the graph resembled a sigmoidal curve. The conductance data was in a general sigmoidal curve equation:
C=Cᵢ+(C_{f}−Cᵢ)/(1+e^{S(t-t}*⁾)
where:
C is current;
Cᵢ is current at t=0;
C_{f} is the final current;
S is a sensitivity constant;
t* is the exposure time required to achieve an inflection point; and
t is the time of exposure.

Referring again to FIG. 8, in step 814, the parameters describing the kinetics of skin conductance changes are calculated. These parameters include, inter alia, skin impedance, the variation of skin impedance with time, final skin impedance, skin impedance at inflection time, final current, exposure time to achieve the inflection time, etc.

In step 816, the skin-permeabilizing device applied in step 808 is terminated when desired values of the parameters describing skin conductance are achieved. For instance, when the skin conductance increases to a certain value, the permeabilizing device may be deactivated. Alternatively, when the rate of change in the value of skin conductance is a maximum, the permeabilizing device may be deactivated. Additional details of the method for regulating the degree of skin permeabilization are disclosed in the aforementioned U.S. application Ser. No. 09/868,442.

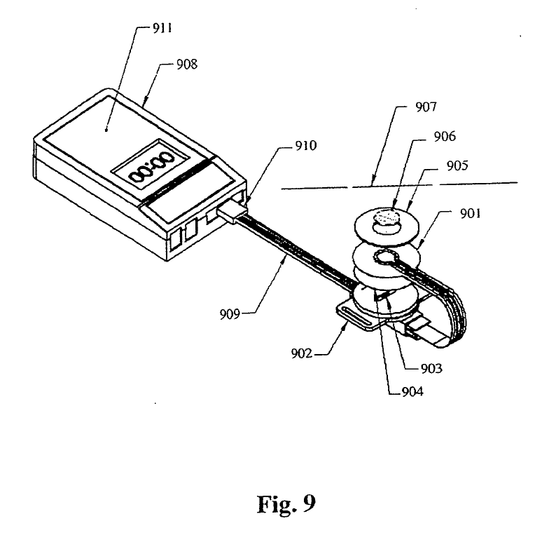

A preferred embodiment of a continuous transdermal glucose monitoring sensor (CGMS) system and method is described in connection with FIGS. 9-11. As discussed above, the term “body fluid” may include blood, interstitial fluid, lymph, and/or analyte. Body fluids include, for example, both complete fluids as well as molecular and/or ionic components thereof. Preferred embodiments of the invention may involve extraction and measurement of just the analyte.

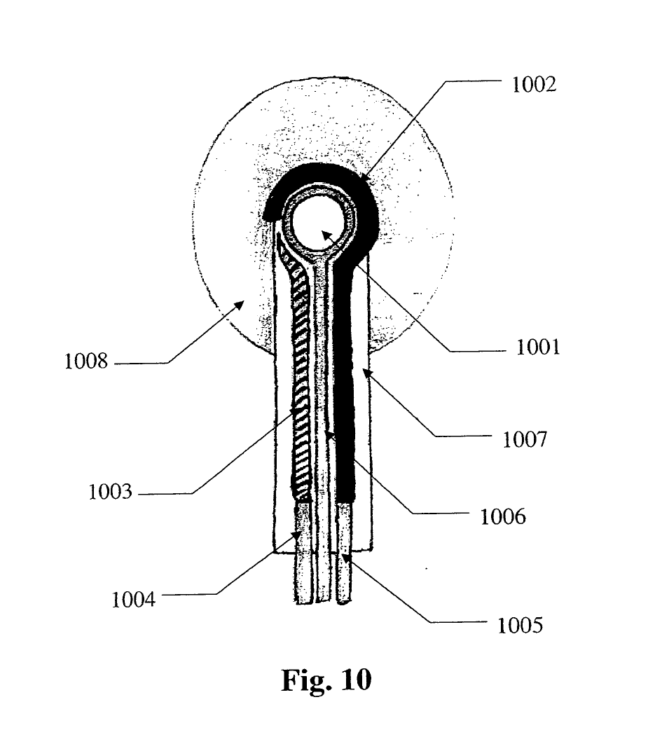

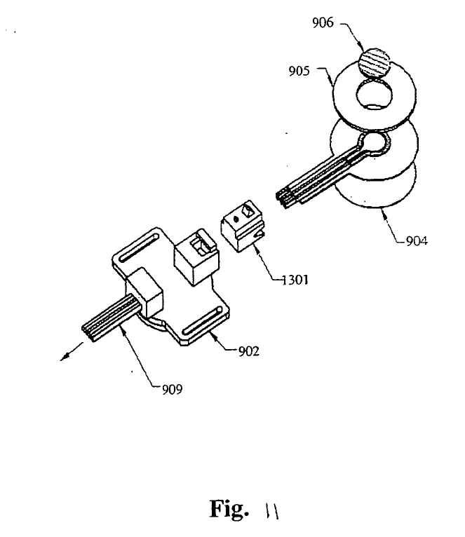

FIG. 9 is a drawing of a continuous glucose monitoring system according to an exemplary embodiment of the invention. In this embodiment, the system includes a sensor assembly generally including a sensor body 901 and a backing plate 902 as well as other components as described herein. The sensor body may include electrodes, as shown in FIG. 10, on its surface for electrochemical detection of analytes or reaction products that are indicative of analytes. A thermal transducer 903, which may be housed in a housing with a shape that corresponds to that of the sensor body 901, is located between the sensor body 901 and the backing plate 902. Electrochemical sensors, such as hydrogen peroxide sensors, can be sensitive to temperature fluctuation. The thermal transducer 903 may be used to normalize and report only those changes attributed to a change in analyte or analyte indicator. An adhesive disc 904 may be attached to the side of the sensor body 901 that faces the thermal transducer 903. An adhesive ring 905 may be attached to the side of the sensor body 901 that is opposite the adhesive disc 904. The cutout center portion of the adhesive ring 905 preferably exposes some or all of the sensor components on the sensor body 901. The adhesive ring 905 and adhesive disc 904 may have a shape that corresponds to that of the sensor body as shown in FIG. 9. A hydrogel disc 906 may be positioned within the cutout center portion of the adhesive ring 905 adjacent a surface of the sensor body 901. During operation, the sensor assembly may be positioned adjacent a permeable region 907 of a user's skin as shown by the dashed line in FIG. 9. The sensor assembly may be attached to a potentiostat recorder 908, which may include a printed circuit board 911, by way of a flexible connecting cable 909. The connecting cable 909 preferably attaches to the potentiostat recorder 908 using a connector 910 that facilitates removal and attachment of the sensor assembly.

The system shown in FIG. 9 can be used to carry out continuous monitoring of an analyte such as glucose as follows. First, a region of skin on the user is made permeable using, for example, sonication as described above. The sensor assembly, such as that shown in FIG. 9, is then attached to the permeable region 907 of skin so that the hydrogel disc 906 is in fluid communication with the permeable skin. An analyte may be extracted through the permeable region 907 of the user's skin so that it is in contact with the hydrogel disc 906 of the sensor assembly. For example, an analyte such as glucose may be transported by diffusion into the hydrogel disc 906 where it can contact glucose oxidase. The glucose can then react with glucose oxidase present in the hydrogel disc 906 to form gluconic acid and hydrogen peroxide. Next, the hydrogen peroxide is transported to the surface of the electrode in the sensor body 901 where it is electrochemically oxidized. The current produced in this oxidation is indicative of the rate of hydrogen peroxide being produced in the hydrogel, which is related to the amount of glucose flux through the skin (the rate of glucose flow through a fixed area of the skin). The glucose flux through the skin is proportional to the concentration of glucose in the blood of the user. The signal from the sensor assembly can thus be utilized to continuously monitor the blood glucose concentration of a user by displaying blood glucose concentration on the potentiostat 908 in a continuous, real-time manner.

Detailed views of a preferred embodiment of the sensor body 901 are shown in FIG. 10. The sensor body 901 includes a body layer 1007 upon which leads 1004, 1005, and 1006 are patterned. The leads may be formed, for example, by coating metal over the body layer 1007 in the desired locations. A working electrode 1001 is typically located at the center of the sensor body 901. The working electrode 1001 may comprise pure platinum, platinized carbon, glassy carbon, carbon nanotubes, mezoporous platinum, platinum black, palladium, gold, or platinum-iridium, for example. The working electrode 1001 may be patterned over lead 1006 so that it is in electrical contact with the lead 1006. A counter electrode 1002, preferably comprising carbon, may be positioned about the periphery of a portion of the working electrode 1001, as shown in FIG. 10. The counter electrode 1002 may be patterned over lead 1005 so that it is in electrical contact with the lead 1005. A reference electrode 1003, preferably comprising Ag/AgCl, may be positioned about the periphery of another portion of the working electrode 1001 as shown in FIG. 10. The electrodes 1001, 1002, and 1003 can be formed to roughly track the layout of the electrical leads 1006, 1005, 1004, respectively, that are patterned in the sensing area of the device. The electrodes 1001, 1002, and 1003 may be screen printed over the electrical leads 1006, 1005, 1004, respectively. The leads can be pattered, using screen printing or other methods known in the art, onto the sensor body 901 in a manner that permits electrical connection to external devices or components. For example, the leads may form a 3× connector pin lead including leads 1004, 1005, and 1006 at the terminus of an extended region of the sensor body as shown in FIG. 10. A standard connector may then be used to connect the sensor electrodes to external devices or components.

The electrochemical sensor utilizes the working electrode 1001, the counter electrode 1002, and the reference electrode 1003 to measure the rate hydrogen peroxide or glucose is being generated in the hydrogel. The electrochemical sensor is preferably operated in potentiostat mode during continuous glucose monitoring. In potentiostat mode, the electrical potential between the working and reference electrodes of a three-electrode cell are maintained at a preset value. The current between the working electrode and the counter electrode is measured. The sensor is maintained in this mode as long as the needed cell voltage and current do not exceed the current and voltage limits of the potentiostat. In the potentiostat mode of operation, the potential between the working and reference electrode may be selected to achieve selective electrochemical measurement of a particular analyte or analyte indicator. Other operational modes can be used to investigate the kinetics and mechanism of the electrode reaction occurring on the working electrode surface, or in electro-analytical applications. For instance, according to an electrochemical cell mode of operation, a current may flow between the working and counter electrodes while the potential of the working electrode is measured against the reference electrode. It will be appreciated by those skilled in the art that the mode of operation of the electrochemical sensor may be selected depending on the application.

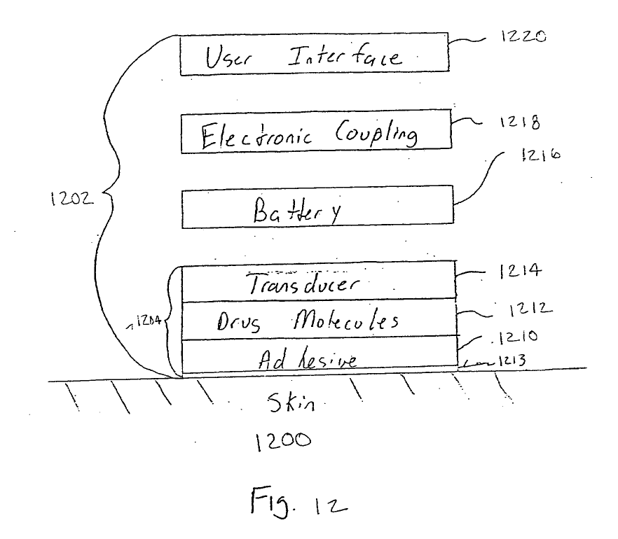

The sensor assembly described generally in relation to FIG. 9 is show in expanded detail from another angle in FIG. 11. The sensor body 901, which is covered on each side by adhesive disc 904 and adhesive ring 905, is shown in relation to the backing plate 902. The hydrogel disc 906 may be positioned in such a manner that it will face toward the user after folding over onto the backing plate 902 as shown in FIG. 9. The sensor body may be connected to the backing plate 902 using standard connectors such as a SLIM/RCPT connector 1301 with a latch that mates with a corresponding connector interface that is mounted onto the backing plate 902.

The sensor assembly shown in FIGS. 9-11 may be incorporated into any one of a number of detection devices. For instance, this sensor assembly may be incorporated into the receiver of FIG. 4 to provide for discrete and/or continuous glucose monitoring. Additionally, the sensor assembly may be connected to a display or computing device through a wireless connection or any other means for electrical connection in addition to the cable 909.

Continuous glucose monitoring as described herein can be achieved without accumulation of a certain volume of body fluid in a reservoir before measuring the concentration of the withdrawn fluid. Continuous glucose monitoring is capable of measuring the blood concentration of glucose without relying on accumulation of body fluids in the sensor device. In continuous glucose monitoring, for instance, one may prefer to minimize accumulation of both glucose and hydrogen peroxide in the hydrogel so that the current measured by the electrochemical sensor is reflective of the glucose flux through the permeable region of skin in real-time. This advantageously permits continuous real-time transdermal glucose monitoring.

Exemplary embodiments of the present invention are also directed to transdermal drug delivery. A drug is defined as a therapeutic, prophylactic, or diagnostic molecule or agent, that may be in a form dissolved or suspended in a liquid, solid, or encapsulated and/or distributed in or within micro or nanoparticles, emulsion, liposomes, or lipid vesicles. Drug delivery is defined as the delivery of a drug into blood, lymph, interstitial fluid, cells, tissues, and/or organs, or any combination thereof.

Referring to FIG. 12, an active patch drug delivery apparatus 1202 that is attached to skin 1200 is depicted. Drug delivery apparatus 1202 includes patch 1204. Patch 1204 includes adhesive 1210, drug molecules 1212 and transducer 1214. Patch 1204 is an active patch. Adhesive 1210 acts as an attaching device. Alternatively, the attaching device may be a vacuum, band, or strap. As transducer 1214 oscillates, the permeability of skin 1200 is increased in accordance with the present invention and drug molecules 1212 are delivered to and/or through skin 1200, or/and after skin 1200 is permeabilized, drug molecules 1212 are transported through skin 1200 to the capillaries and blood vessels below skin 1200. A limiting step membrane 1213 may be located between skin 1200 and drug molecules 1212.

Transducer 1214 preferably operates at a frequency in the range of between 20 kHz to 2.5 MHz, using appropriate electrical signal generators and amplifiers. Transducer 1214, more preferably, is operating at a frequency in the range of between 20 and 200 kHz. Other ultrasound parameters include, but are not limited to, amplitude, duty cycle, distance from the skin, coupling agent composition, and application time and may be varied to achieve sufficient enhancement of transdermal transport. The intensity preferably varies from 0 to 20 W/cm². Further, transducer 1214 may be configured as a cylinder, a hollow cylinder, a hemispherical configuration, conical configuration, planer configuration or rectangle configuration. Transducer 1214 may also consist of an array of acoustic elements that are swept in time. Transducer 1214 may be comprised of quartz, PVDF, ceramic including PZT and screen printed ceramic, magnetostrictive, or composite material including molded ceramic and benders. Transducer 1214 may be used alone, or in conjunction with other forces, or contributors, to enhance drug delivery. These other forces, or contributors, include, but are not limited to, a magnetic field including electromagnetic forces, an electrical current or iontophoresis, mechanical skin manipulation, chemical enhancement, heat, and osmotic forces.

Transducer 1214 administers ultrasound preferably at frequencies of less than or equal to about 2.5 MHz, preferably at a frequency that is less than 1 MHz, and more typically in the range of about 20 to 100 kHz. Exposures to ultrasounds from transducer 1214 are typically between about 5 seconds and about 10 minutes continuously, but may be shorter and/or pulsed, for example, at 100 to 500 msec pulses every seconds for a time sufficient to permeabilize the skin. The ultrasound intensity is of a level that preferably does not raise skin 1200's temperature more than about 1 to 2 degrees Centigrade and does not cause permanent damage to the skin. The intensity typically is less than 20 W/cm², preferably less than 10 W/cm². Intensity in time of application is inversely proportional to exposure time, so that high intensities are applied for shorter period of times in order to avoid skin damage. It should be noted that although normal low range ultrasound is 20 kHz, comparable results may be achieved by varying the frequency to less than 20 kHz, or into the sound region.

The time needed for permeabilization is dependant upon the frequency and intensity of the ultrasound from transducer 1214 and the condition of skin 1200. For example, at a frequency of 20 kHz, an intensity of 10 W/cm², and a duty cycle of 50 percent, skin 1200 is permeabilized sufficiently in about 5 minutes if skin 1200 is on a human forearm.

Permeabilizing ultrasound may be applied for a predetermined amount of time or may be applied only until permeabilization is attained. Because skin 1200 characteristics or properties may change over time, based on aging, diet, stress, and other factors, it may be preferable to measure permeability as ultrasound is applied to minimize the risk of skin 1200 damage. Several methods may be used to determine when sufficient permeabilization has been reached. One method measures relative skin conductivity at the permeabilization site versus a reference point. These measurements are performed by applying a small AC or DC electric potential across two electrically isolated electrodes in contact with skin 1200. Electric current flowing through these electrodes is measured using an ammeter and skin 1200 resistance is measured using the values of the potential and current. Drug delivery patch apparatus 1202 may serve as one of the electrically isolated electrodes in contact with skin 1200. Preferably, drug delivery patch apparatus 1202 permeabilizes skin 1200 prior to the conductivity tests.

Another way to determine when sufficient permeabilization has been reached is to measure the conductivity of skin. Fully permeabilized skin has a resistance of no more than about 5 kilo-ohms (kΩ) measured across approximately 1.7 cm². Another method is to detect and/or quantify the transdermal movement of an analyte, such as creatinine, calcium or total ions, that is present in interstitial fluid in a fairly constant amount, and may be used either to calibrate the concentration of analyte to be extracted and quantified, or as a measure of permeabilization. The higher the constant analyte flux, the greater degree of permeabilization. The degree of permeability also may be monitored using a sensor attached to drug delivery patch apparatus 1202 that determines the concentration of drug molecules 1212 being delivered or an analyte being extracted. As the permeability increases, the drug concentration within drug delivery patch 1202 decreases.

Drug delivery patch apparatus 1202 also may be applied to pretreated skin 1200. In other words, permeabilization of skin 1200 is already achieved. Drug delivery patch apparatus 1202 is placed over pretreated skin 1200 to deliver drug molecules 1212. Any known device may be used to pre-treat skin 1200, including, but not limited to, devices that apply physical forces, chemical forces, biological forces, vacuum pressure, electrical forces, osmotic forces, diffusion forces, electromagnetic forces, ultrasound forces, cavitation forces, mechanical forces, thermal forces, capillary forces, fluid circulation across the skin, electro-acoustic forces, magnetic forces, magneto-hydrodynamic forces, acoustic forces, convective dispersion, photo-acoustic forces, by rinsing body fluid off skin, and any combination thereof.

Drug molecules 1212 may include a variety of bioactive agents, such as proteins, peptides, viruses, nucleic acids (DNA, RNA, RNAi, aptamers, oligonucleotides), saccharides and polysaccharides, for example. General classes of suitable bioactives may include, for example, childhood and traveler's vaccines (tetanus, diphtheria, mumps, influenza, mumps, measles, rubella, hepatitis, etc.), therapeutic proteins, and synthetic organic and inorganic molecules such as anti-inflammatories, anti-virals, anti-fungals, antibiotics, anesthetics and analgesics. The bioactive agent may have a local effect (such as in a local anesthetic) or a systemic effect (such as in a vaccine), depending on the specific application. In one example, lidocaine may be utilized as the drug to achieve rapid topical anesthesia. Drug molecules 1212 may be administered in an appropriate pharmaceutically acceptable carrier having an absorption coefficient, similar to water, such as aqueous gels, ointment, lotion, or suspension. The drug molecules may also be delivered in a pharmaceutically acceptable carrier that is hydrophobic, such as in a drug-containing dispersion, cream or emulsion. Drug molecules 1212 also may be contained in an adhesive 1210 that attaches to skin 1200. Further, drug molecules 1212 also may be encapsulated or suspended in a liquid, gel, or solid matrix within patch 1204. Additionally, the bioactive molecule may also be delivered to the permeated area in a liquid reservoir that is contained in an adhesive “bubble” pocket patch.

Drug delivery patch apparatus 1202 also includes a battery 1216. Battery 1216 acts as a power source for transducer 1214. Battery 1216 provides a relatively high-energy burst. Drug delivery patch apparatus 1202 also includes electronic coupling 1218 that serves as the drive electronics for drug delivery patch apparatus 1202. Drug delivery patch apparatus 1202 also includes user interface 1220.

In one embodiment, patch 1204 includes transducer 1214, drug molecules 1212, and adhesive 1210. In another embodiment, patch 1204 includes transducer 1214, drug molecules 1212, adhesive 1210, battery 1216, electronic coupling 1218, and user interface 1220. In another embodiment, patch 1204 includes transducer 1214, drug molecules 1212, adhesive 1210, and battery 1216. In another embodiment, adhesive 1210 is to the side of transducer 1214 and drug molecules 1212.

Battery 1216, electronic coupling 1218, and user interface 1220, may be located elsewhere on a user and in communication with patch 1204 via hard wire or telemetry. In another embodiment, user interface 1220 may be located elsewhere on the user and is in communication with patch 1204 via hard wire, telemetry, infrared, or fiber optic means. Thus, the elements of drug delivery apparatus 1202 may be detachable and portable from each other. Further, any of the components of drug delivery apparatus 1202 may be disposable or reusable. For example, patch 1204, which includes transducer 1214, drug molecules 1212 and adhesive 1210, may be disposed after detachment from skin 1200. However, battery 1216, electronic coupling 1218, and user interface 1220 may be re-usable with further patches 1204.

In one embodiment, transducer 1214 operates alone to push drug molecules 1212 through and to skin 1200. Alternatively, drug delivery patch apparatus 1202 and transducer 1214 may operate in conjunction with a driving force that further facilitates the transdermal transport of drug molecules 1212. These forces include, but are not limited to physical forces, chemical forces, biological forces, vacuum pressure, electrical forces, osmotic forces, diffusion forces, electromagnetic forces, ultrasound forces, cavitation forces, mechanical forces, thermal forces, capillary forces, fluid circulation across the skin, electro-acoustic forces, magnetic forces, magneto-hydrodynamic forces, acoustic forces, convective dispersion, photo-acoustic forces, by rinsing body fluid off skin, and any combination thereof.

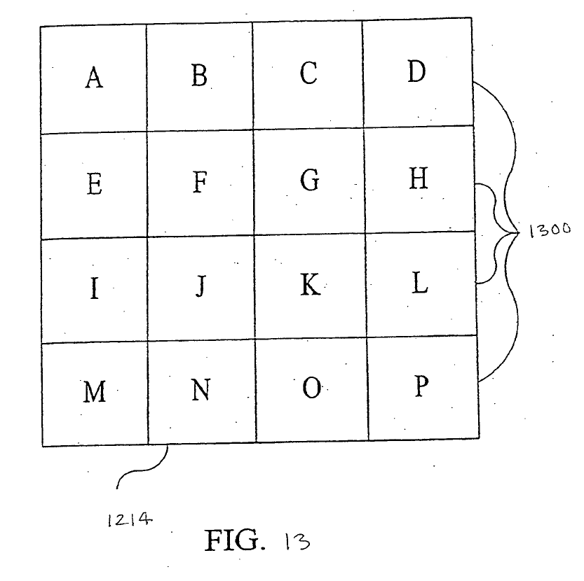

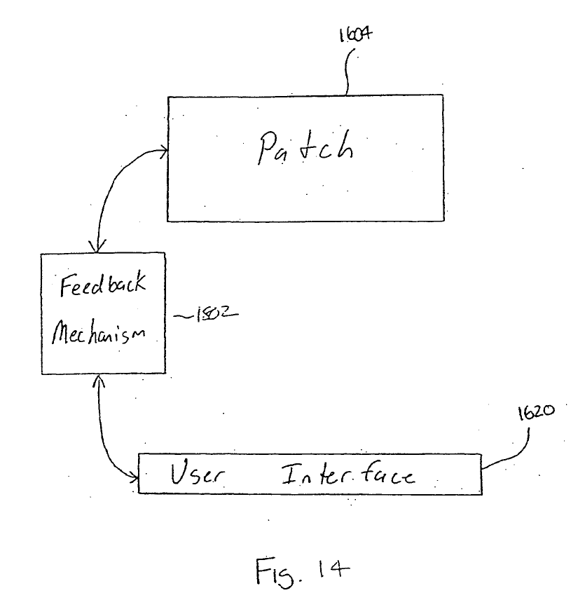

Referring to FIG. 13, an embodiment of transducer 1214 is depicted. Transducer 1214 may be an array of acoustic elements that are swept in time as ultrasound is applied to drug molecules 1212, and through adhesive 1210 to skin 1200. Acoustic elements 1300 comprise transducer 1214. Elements 1300 are depicted as squares within a larger square. Elements 1300 are not limited to this configuration and may be configured as a cylinder, a hollow cylinder, hemispherical, conical, planer, or rectangular. Each acoustic element of elements of 1300 may be swept individually or within a group as transducer 1214 is activated. For example, element A activates, followed by elements B and E, then followed by elements C, F, and I, and so on. Element P may be activated last as transducer 1214 is swept. Further, acoustic elements 1300 may comprise fingers. Referring to FIG. 13, a finger may be depicted as elements A, E, I, and M. Each finger may be activated or swept in time. Acoustic elements 1300 may be configured to channel the ultrasound energy from transducer 1214 to a specified area in 100 smaller than the area of transducer 1214.

Referring to FIG. 14, patch 1204 and user interface 1220 are coupled to feedback mechanism 1402. Feedback mechanism 1402 may be detachable from user interface 1220. Alternatively, feedback mechanism 1402 may be contained within user interface 1220. Thus, feedback mechanism 1402 may be contained within drug delivery patch apparatus 1202. Feedback mechanism 1402 provides for programming of drug delivery rates or pre-set doses of drug molecules 1212. Feedback mechanism 1402 also may provide memory to record or display historical delivery data to user interface 1220. Feedback mechanism 1402 communicates the on time of transducer 1214 to user interface 1220 for display to the user. Feedback mechanism 1402 also may provide alarms for low drug molecules 1212 and/or low power in battery 1216. Thus, feedback mechanism 1402 alerts a user via a user interface 1220 that drug molecules 1212 and patch 1204 needs to be replenished or that drug delivery patch apparatus 1202 is low on power.

Feedback mechanism 1402 also may monitor the amount of drug molecules 1212 delivered via transdermal transport. Feedback mechanism 1402 also may monitor the amount of ultrasonic energy, or other driving forces listed above, applied to skin 1200 by transducer 1214. Limits may be set in feedback mechanism 1402 to limit the ultrasound energy from transducer 1214 so as to not irritate or damage skin 1200. Feedback mechanism 1402 also may monitor the concentration of drug molecules 1212 remaining in patch 1204. Feedback mechanism 1402 also may monitor the concentration of drug molecules or analytes in the interstitial fluid, blood, and other body fluids. Feedback mechanism 1402 also may monitor the amount of cavitation produced by the application of ultrasound energy. Feedback mechanism 1402 also may monitor the degree of physiological effects such as blood pressure, EMG, EEG, and ECT feedback in order to measure delivery of drug molecules 1212. Feedback mechanism 1420 also may provide connections with additional patches or testing devices in order to perform conductivity tests.

In another embodiment, a local anesthetic, e.g. lidocaine, is applied to a site that has been made permeable. For instance, a topical solution of 4% lidocaine may be applied to a site that has been treated with a SonoPrep® device. Local anesthesia delivered in this manner has been shown to shorten the onset of anesthesia from 60 minutes to 5 minutes. This technique may be applied where local anesthesia is desired, such as prior to IV insertions, blood draws, or other needle sticks. The components necessary for a particular procedure may be packaged in a tray for clinical use. Such tray may include an ultrasonic coupling medium cartridge, disinfectant cartridge, one or more target rings, injection site marker, and a skin prep pad.

According to another aspect of the invention, vaccines can be administered with enhanced transdermal transport. Generally, vaccines are administered for the prevention, amelioration or treatment of infectious or cell-mediated diseases. Prophylactic vaccines are commonly used to provide protective immunity from diseases such as influenza, poliomyelitis, varicella zoster (chicken pox), and measles, as well as several other diseases. Therapeutic vaccines are used to generate cell-mediated immune responses to treat clinically indicated HPV, HIV, cancer, etc. Immunotherapeutics to treat autoimmune diseases such as psoriasis, etc. are also included in this category.

Immunization is the process of causing immunity by injecting antibodies or provoking the body to make its own antibodies against a certain microorganism. Immunization may be a result of a vaccination.

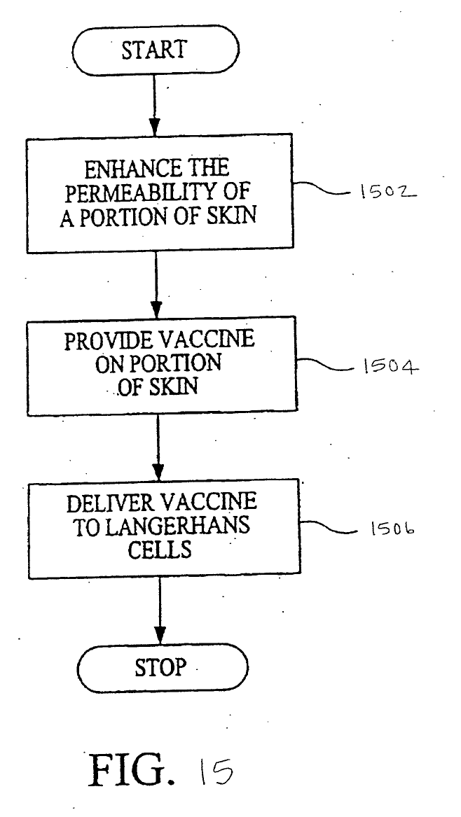

FIG. 15 depicts a method for transdermal vaccination by sonophoresis according to one embodiment of the present invention. Referring to FIG. 15, in step 1502, the permeability of the skin is increased. This may be achieved by several methods, including those discussed above.

In one embodiment, ultrasound may be applied at about 10 W/cm², with a duty cycle of about 50%. Ultrasound may be applied at a distance from the skin of about 0.5 mm to 1 cm, and for an application time of from about 30 seconds to about 5 minutes.

A coupling medium may be used between the transducer and the skin, and may contain aqueous or non-aqueous chemicals including, but not limited to, water, saline, alcohol, including ethanol and isopropanol (1-100% mixtures with saline), surfactants, fatty acids such as linoleic acid (0.1-2% mixtures in ethanol-water (50:50) mixture), azone (0.1-10% mixtures in ethanol-water (50:50) mixture), 01-50% polyethylene glycol in saline, 1-100 mM EDTA, EGTA, or 1% SLS and silica particles. The coupling media provide effective transfer of ultrasound energy from transducer to the skin. Appropriate mixtures of these coupling media may also enhance cavitation activity inside, on the surface, or near the skin, thus inducing more effective transport of molecules across the skin.

In step 1504, after the permeability of the skin is increased, sonication is terminated, and a vaccine is provided on the permeated skin. In one embodiment, the vaccine may be incorporated into a transdermal patch. Other forms of the vaccine, such as gels and liquids, may also be used.

The vaccine may comprise as the active ingredient a peptide, protein, allergen, or other antigen, or DNA encoding any of the foregoing and may also include other adjuvants normally employed. These vaccines may be used as prophylactics as in tetanus toxoid, measles, mumps, hepatitis (A-C) and therapeutics such as in immunotherapeutics to treat various forms of cancer.

In step 1506, the vaccine is delivered to the skin cells. In one embodiment, the vaccine is delivered to skin cells, including Langerhans cells, dendritic cells, and keratinocytes. Once the vaccine is received by the skin cells, the vaccine is transported to the lymph nodes efficiently, increasing the efficiency of vaccination.

In another embodiment, the vaccine is transported transdermally through, in, or into the skin and into the bloodstream, wherein it acts as if it were injected in a conventional manner.

In another embodiment of the present invention, the vaccine is provided simultaneously with the application of ultrasound. The ultrasound in this embodiment is used both to permeabilize the skin, as well as and to deliver the vaccine transdermally to the Langerhans cells. The ultrasound acts as a driving force. Examples of using ultrasound to transport drugs from a patch are discussed above.

In another embodiment of the present invention, ultrasound is applied to the skin to increase the permeability of the skin. Once the vaccine is provided, additional driving forces are provided to deliver the vaccine to the body. Examples of driving forces include, inter alia, physical forces, chemical forces, biological forces, vacuum, electrical forces, osmotic forces, diffusion forces, electromagnetic forces, ultrasound forces, cavitation forces, mechanical forces, thermal forces, capillary forces, fluid circulation across the skin, electro-acoustic forces, magnetic forces, magneto-hydrodynamic forces, acoustic forces, convective dispersion, photo acoustic forces, and any combination thereof.

According to another aspect of the invention, a step of skin hydration may be employed prior to or concurrently with increasing the porosity of the skin (e.g. by applying ultrasound) to improve the transdermal transport across a biological membrane. The hydration can be utilized in connection with the other methods described herein including continuous transdermal analyte monitoring, transdermal drug delivery, transdermal delivery of anesthetic or transdermal delivery of a vaccine. Skin hydration prior to or concurrently with increasing the porosity when used with transdermal analyte monitoring for skin hydration prior to attaching the sensor may improve sensor performance by establishing or stabilizing liquid pathways between the skin and the sensor, improving the moisture balance over the sensor-skin interface, and/or continuing to maintain ample water at the hydrogel to maintain enzyme activity. The skin hydration procedure can be performed, for example, by applying a hydrating agent to the target skin site. The hydrating agent may be applied in combination with a delipidation or cleansing agent. Where both hydrating and cleansing agents are utilized, they may be applied in a single application using a single solution. Alternatively, the cleansing agent and the hydrating agent can be applied using successive application of two different solutions. In one aspect, one or both solutions are applied using a pad applicator. In another aspect, the solution can be held in contact with the skin by positioning it in the bellows of a sonication device or another device that functions to hold a liquid in contact with skin.

In one embodiment, a glycerin/water prep pad solution may be prepared for skin hydration. The following batch formulation can be used to prepare the glycerin/water prep pad solution. Glycerin 99% USP (300.00 grams) is added to a first container. Nipagin M (i.e., methylparaben) (2.70 grams), Nipasol M (i.e., propylparaben) (0.45 grams), and benzyl alcohol NF (30.00 grams) are dissolved in a second container and then added to the first container. The glycerin and benzyl alcohol solutions are then mixed in the first container until the solution clears. Deionized (1133.85 grams) water is then added to the solution in the first container and mixed until homogeneous. Potassium Sorbate NF (1.50 grams) is added to the solution in the first container and mixed until homogeneous. Glydant® 2000 (1.50 grams) is then added to the solution in the first container and mixed until homogeneous. Lastly, deionized water (30.00 grams) is added to the solution in the first container and mixed until homogeneous.

In one embodiment, a 1 3/16″ prep pad is utilized. Preferably the prep pads are composed of 70% polypropylene/30% cellulose. In one embodiment, the prep pad has a width that ranges from 1 1/16″ to 1 5/16″. In one embodiment, the thickness of the prep pad is 21-29 mils. In another embodiment, the thickness of the prep pad is 26-34 mils. In one embodiment the prep pad has a basis weight of 1.43-1.87 g/yd using ATM#102. In another embodiment, the prep pad has a basis weight of 1.72-2.24 g/yd using ATM#102. Preferably, the prep pad is utilized with a prep pad solution, such as the prep pad solution described above, to hydrate a biological membrane before increasing its porosity. The prep pad may be utilized with any of the solutions for increasing transdermal transport described within this application.

In one embodiment, a method to treat skin prior to sonopermeation of the stratum corneum is disclosed. Sonopermeation may be performed, for example, using the device described in connection with FIG. 2 or with a SonoPrep® device available from Sontra Medical Inc. of Franklin, Mass. The method may comprise the following steps: (a) delipidation followed by hydration of the skin using a single solution, or (b) delipidation followed by hydration of the skin using two separate solutions; and (c) sonication of the site.

The solution described in (a) may include, for example, a combination of hydrating and delipidation (cleansing) agents such as potassium lauryl sulfate, polysorbate 20, tetrasodium EDTA, vitamin E acetate and aloe to accomplish delipidation followed by hydration in a single step. Other combinations may include soy lecithin and isopropyl alcohol in various proportions. Among these examples, Potassium lauryl sulfate, isopropyl alcohol, tetrasodium EDTA are delipidation agents and vitamin E acetate, aloe and soy lecithin are lipid soluble permeation enhancers. These delipidation agents and permeation enhancers may be dissolved in a water-based solvent with hydrating agents such as polysorbate 20 and glycerol to provide a combined delipidation/hydration composition. In one embodiment, the lipid solubilizer and hydrating molecules can be applied in a single step, such as in a mixture of alcohol and glycerol.

Alternatively, the delipidation of the stratum corneum and hydration may be accomplished in two separate steps as in alternative (b) above. The solutions may include an alcohol delipidation agent, such as isopropanol, to remove the skin oils and an amphiphilic hydrating agent, such as glycerol, that is capable of permeating the skin. Other skin hydrating agents may include the polyethylene glycols and polysorbates. The amphiphilic character of an aqueous glycerol solution enables permeation of the lipid bilayer physiology of the skin assisting in uniform hydration of the treated site.

Hydration of the stratum corneum can lead to significant changes in its barrier properties. Hydration can lead to swelling of corneocytes and expansion of the intercellular lipid lamellae, leading to enhanced fluidity and loosening of the lipid “mortar” of the skin's “brick-and-mortar” arrangement, thereby preparing the skin for drug delivery, for example. Described herein are methods to desolvate (delipidize) and re-hydrate the skin prior to cavitation-induced permeation of the site by low frequency ultrasound, which may be carried out, for example, with the SonoPrep® device available from Sontra Medical, Inc. These methods, according to exemplary embodiments of the invention, can provide higher percentages of successful sonications measured by skin impedance, sonication curves, and pain/discomfort scores. It is hypothesized that this skin pretreatment method uniformly hydrates the epidermal lipid lamellae to enable reproducible and painless ultrasound-induced skin permeation. Mechanistically, it is hypothesized that higher mobility of hydrated corneocytes and lamellae provides ease of poration by a cavitating liquid without heat buildup due to immobility of the skin components.

In one embodiment, the solvation and removal of surface lipids of the stratum corneum can be accomplished by use of an alcohol wipe (e.g., 70% isopropanol), cholesterol derivatives such ascholesteryl sulfate, cholic acid, glycocholic acid, taurocholic acidursodeoxycholic acids, fatty acid derivatives such a sodium dodecyl sulfate, potassium dodecyl sulfate, CHAPS, CHAPSO, cetyl triammonium bromide (CTAB) and micelle-forming amphiphilic polymers such as polyethylene glycols, Triton X-100, pluronics, Tweens, etc. and combinations thereof. The hydration and swelling of the skin lamellae can be achieved by application of hydrating agents that include water, glycerol, tweens, and hyaluronic acids, for example.

In another embodiment, the lipid solubilizer (delipidation agent) and the hydrating fluid can be combined in the cavitation fluid (in the bellows) to porate the stratum corneum by ultrasound-induced cavitation using a device such as the SonoPrep® device. For example, a bellows cartridge can be used to inject 5 ml of fluid between the ultrasound transducer and the skin. The fluid establishes cavitation which ultimately porates the skin. A bellows configuration is used such that the fluid can be automatically picked back up off the skin and disposed once sonication is complete. The cavitation fluid may comprise a coupling medium, such as saline and 1% sodium lauryl sulfate (SLS), combined with either a lipid solubilizer (dilipidation agent) or hydration agent or both.

According to another embodiment of the invention, a vacuum procedure can be used to enhance transdermal transport. For instance, the following vacuum procedure may be applied: (1) sonicate the intact skin, (2) apply a glass chamber over the sonicated site, (3) cover the top of the chamber and connect the chamber to a vacuum pump, (4) draw a negative pressure (e.g., 4 psig) on the sonicate site for 10 minutes, and (5) remove the glass chamber. After removing the glass chamber, a sensor (e.g., a glucose sensor) or patch (e.g., a drug delivery patch) may be attached to the sonicated site.

In another embodiment, the skin hydration procedure can be conducted by soaking the target skin site with water, electrolyte solution, or other types of solutions or agents, which may help to adjust moisture level between the target skin site and the hydrogel. After the procedure, the skin sites may be thoroughly rinsed with water and dried before sensor or patch placement.

### EXAMPLE 2

In this example, sonication parameters were evaluated to identify a desirable skin pretreatment agent.

This experiment entailed screening of skin treatment agents to enable reproducible and painless ultrasound-induced skin permeation. The agents tested were isopropanol, Lippo gel (Hawkins Pharmaceuticals, Inc., Minneapolis, Minn.), phosphate buffered saline (PBS), pyrrolidone carboxylate (Sigma, Inc.), taurocholic acid (sodium taurocholate, Spectrum Chemicals, Gardena, Calif.), and glycerol (Spectrum Chemicals, Gardena, Calif.). Among these, isopropanol, pyrrolidone carboxylate and taurocholic acid served as delipidation agents and phosphate buffered saline and glycerol served as hydrating agents. Lippo Gel is a commercially available skin permeation enhancer with lipid dissolution and skin hydrating agents.

The agents were dissolved in deionized water in the following concentrations: Isopropanol (70% weight/volume (w/v)), pyrrolidone carboxylate (1% w/v), sodium taurocholate (3% w/v) and glycerol (5% w/v). Surgical gauze pads were wet with the solutions. The pads were wiped over the treatment site 5 times. Sonication parameters such as skin impedance, pain scoring, and sonication curves were recorded. A “sonication curve” may be generated from an in-process measurement of skin conductance during sonication. The sonication is terminated when the skin reaches a pre-programmed conductance. Healthy volunteers between the ages of 20-60 were enrolled in the study. The control groups received no skin pretreatment.

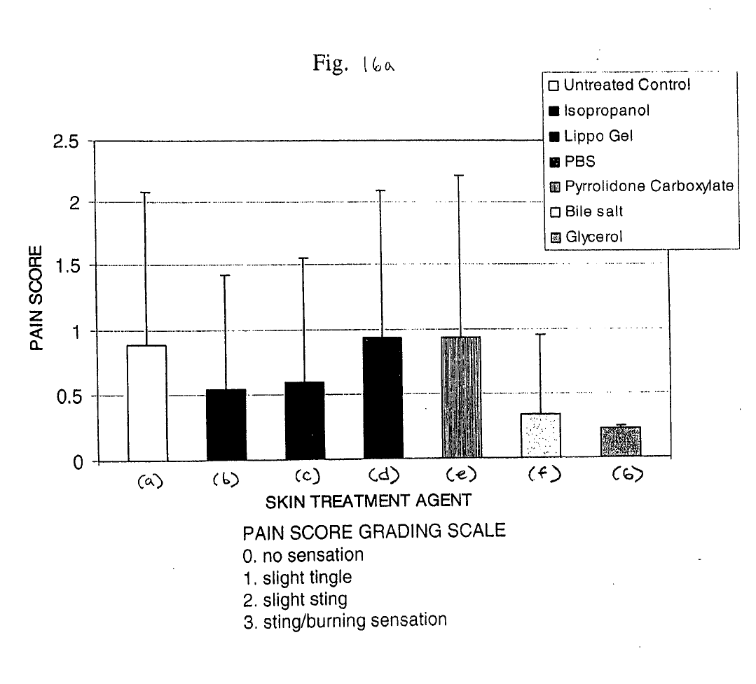

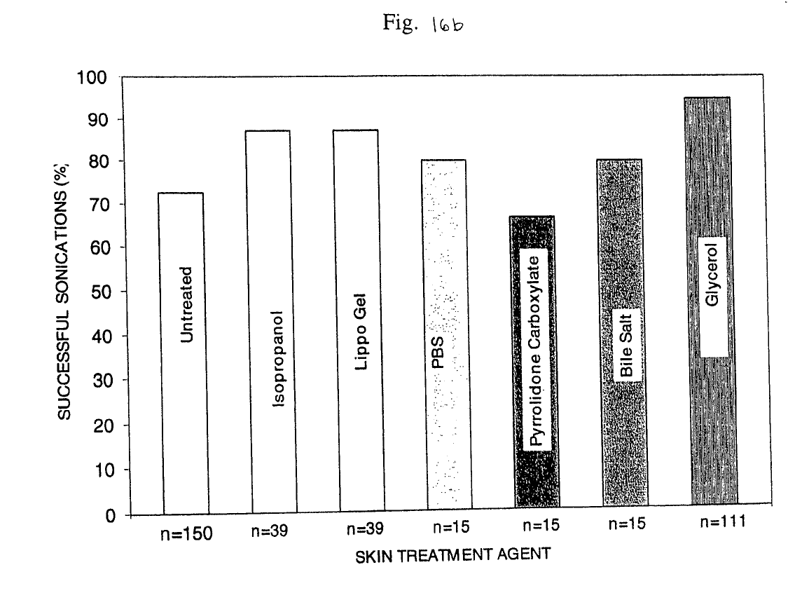

FIG. 16a is a graph of the pain/discomfort score plotted as a function of skin pretreatment agent. The pain/discomfort score represents the pain or discomfort reported by member of the tested group during sonication and was defined as: 0=no sensation, 1=slight tingle, 2=slight sting, and 3=sting/burning sensation. The groups tested were (a) untreated control, (b) isopropanol, (c) Lippo gel, (d) PBS, (e) pyrrolidone carboxylate, (f) bile salt, and (g) glycerol. The data in FIG. 16a show that pain scores were lowest for groups receiving glycerol, isopropanol, and bile salt as skin pretreatment modalities. FIG. 16b is a graph of the percent success rate of sonication by a SonoPrep® device plotted as a function of skin pretreatment for the same groups shown in FIG. 16a. A successful sonication is defined as one that achieves a change in skin conductance greater than 10 μ-amperes or a skin impedance less than or equal to 10 k-ohms. Additionally, successful sonication causes no bruising or welting of the treated site. FIG. 16b shows that the percent of successful sonication was highest for glycerol pretreatments. The untreated controls had success rates of about 70%.

### EXAMPLE 3

This example involved a method to solvate and strip skin surface lipids using a liposoluble solvent such as alcohol followed by hydration of the epidermal corneocytes using a hydrating solvent such as glycerol. This example demonstrated that the sequence of the lipid stripping followed by the skin hydration step may be important.

Human volunteers between the ages of 20-60 were enrolled in the study. The sites of treatment were the dorsum of the hand and the anticubital of the arm.

Sonication on skin pre-treated with an alcohol wipe followed by a glycerol wipe, an alcohol wipe followed by a baby wipe, and a baby wipe alone were evaluated in this example. The alcohol wipe used contained 70% isopropanol in deionized water. Pre-packaged alcohol wipes were used for the study. The baby wipes used in the study were commercially available under the Huggies® brand name. A 5% w/v solution of pharmaceutical grade glycerol (Spectrum Chemicals, Gardena, Calif.) in sterile filtered, deionized water (Spectrum Chemicals, Gardena, Calif.) was used for the study. Sonication by a SonoPrep® device followed each skin treatment method. The control group subjects had no skin pretreatment prior to sonication.

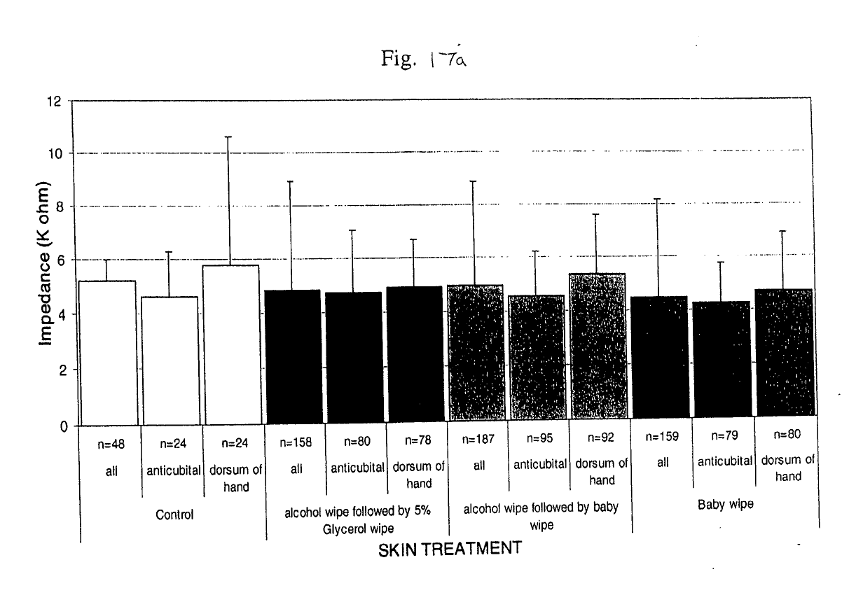

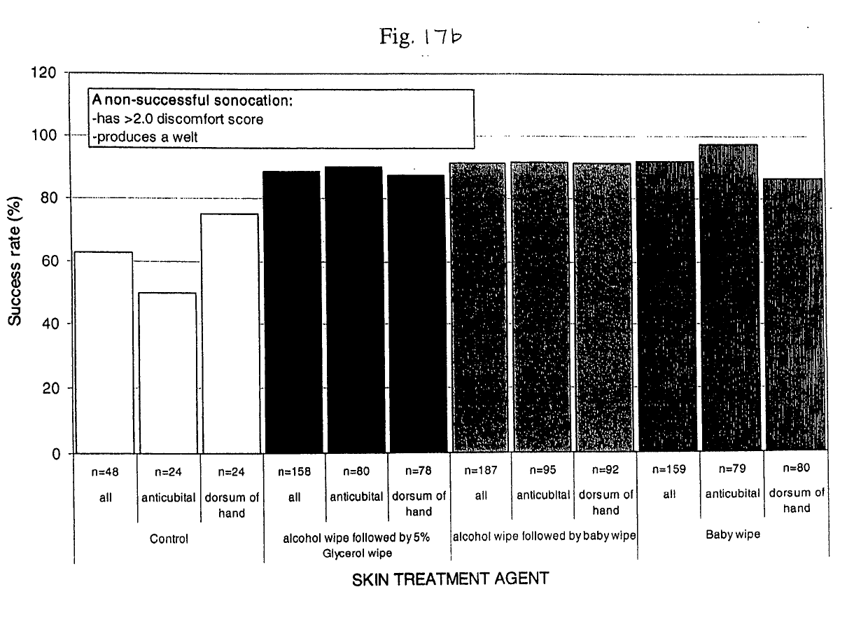

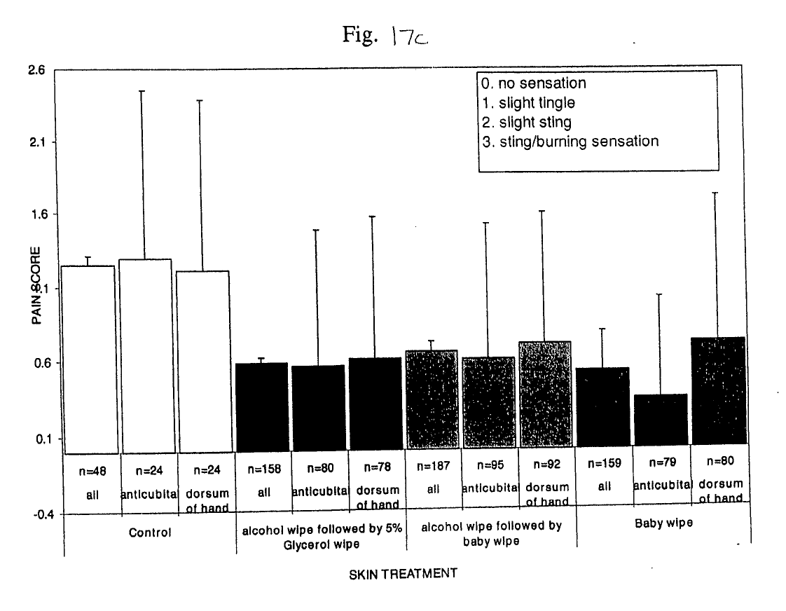

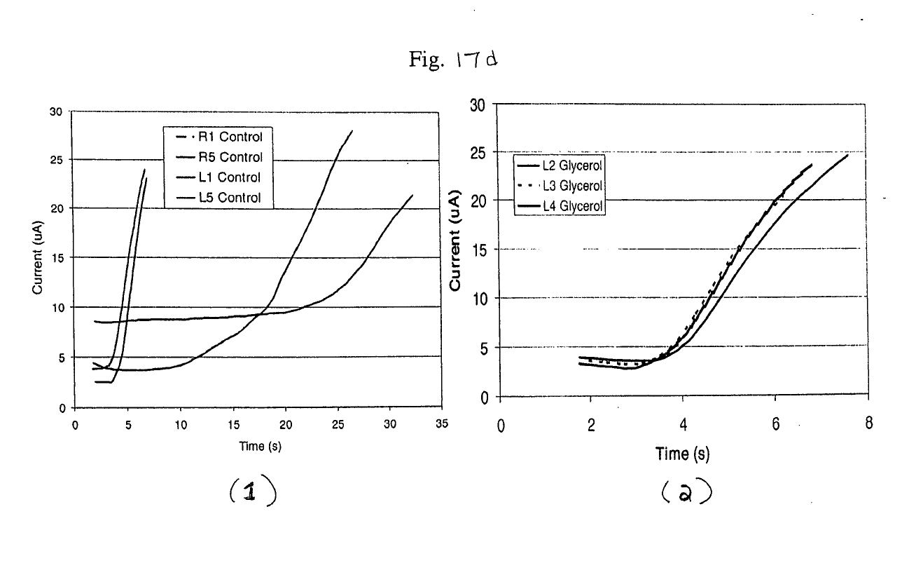

FIG. 17a is a graph of skin impedance obtained on ultrasonicated skin (dorsum, anticubital) as a function of skin pretreatment. The three pretreatments shown in FIG. 17a are (a) control (no hydrating treatment), (b) alcohol (70% w/v isopropanol, IPA) wipe, followed by a 5% w/v glycerol wipe, (c) an alcohol wipe followed by a baby wipe, and (d) a baby wipe alone. Data in FIG. 17a demonstrates that the skin barrier function, indicated by inherent impedance (in k-ohms) of the skin, can be successfully disrupted post-sonication by skin pretreatment and sonication for all sites tested (anticubital, dorsum) for all three pre-treatments tested. Skin impedance was measured by a PrepCheck® impedance measurement device available from Sontra Medical, Inc.

FIG. 17b is a graph of percent success rate of sonication by a SonoPrep® device obtained in human volunteers. A successful sonication is one that achieves: (a) a skin impedance less than or equal to 10 k-ohms, as measured by a PrepCheck impedance measurement device and (b) pain/discomfort scores <2 on a pain scale, with no bruises and welts caused by the ultrasound sonication process. The sonication process, recorded during sonication, is plotted as the conductance of the skin (in μ-amperes) over sonication time (in seconds). A change in skin conductance (A conductance) of ≧15 μ-amperes demonstrates that the barrier function of the stratum corneum has been breached. Through confocal microscopy studies on skin sonicated under these conditions, it has been demonstrated that micron-sized pores or channels are created to allow the transit of molecules through the skin. Additionally, it was shown that skin sonicated under these conditions had significant transepidermal water loss (TEWL), indicating that the stratum corneum had been breached. Conversely, a non-successful sonication is defined as one that has a pain/discomfort score >2. The groups shown in FIG. 17b were the same as those shown in FIG. 17a. The sites tested were on the dorsum and the anticubital. Data shown in FIG. 17b demonstrates significantly higher rates of successful sonication for the skin pretreatment groups, as compared to the untreated control groups.

FIG. 17c is a graph of average pain/discomfort score associated with ultrosonication by SonoPrep® in human volunteers. The pain/discomfort score represents the pain or discomfort reported by member of the tested group during sonication and was defined as: 0=no sensation, 1=slight tingle, 2=slight sting, and 3=sting/burning sensation. The groups shown in FIG. 17c were the same as those shown in FIG. 17a. The sites tested were on the dorsum and the anticubital. Data in FIG. 17c demonstrated that the discomfort associated with sonication for the skin pretreatment groups was significantly lower than the untreated controls.

FIG. 17d(1)-(2) show that sonication can be achieved in a successful and reproducible manner when skin is pretreated with an alcohol wipe (70% isopropanol) followed by a glycerol wipe (5% glycerol). Both FIG. 17d(1)-(2) are graphs current (μ-amperes of conductance) during sonication using SonoPrep® as a function of sonication time (seconds). The time required to reach a specified current value can be interpreted as the time required to achieve sonication by SonoPrep®. Specifically, FIG. 17d(1) shows sonication by SonoPrep® without any pre-treatment on four sites (R1, R5, L1, L5; R denotes the right anticubital, L denotes the left anticubital), while FIG. 17d(2) represents sonication by SonoPrep® with pre-treatment by an alcohol wipe (70% isopropanol), followed by a 5% glycerol wipe on three sites (L2 Glycerol, L3-Glycerol, L4-Glycerol), all on the left anticubital. The data of FIG. 17(d)(1)-(2) demonstrate that skin pretreated with an alcohol (70% Isopropanol) wipe, followed by a 5% w/v glycerol wipe resulted in successful and reproducible sonication by SonoPrep®.

### EXAMPLE 4

In this example, sonication on skin pre-treated with an alcohol wipe followed by glycerol mixed in with the ultrasonication cavitation fluid (sodium lauryl sulfate) was evaluated. This example describes another method to accomplish skin delipidation and subsequent hydration. This method comprised an initial delipidation step (alcohol wipe) followed by combined delipidation/hydration/cavitation accomplished during ultrasonication. Data with varied concentrations of glycerol in the bellows (5-20% w/v) was also collected to determine a desired glycerol concentration. As mentioned earlier, the bellows also contained 1% w/v sodium lauryl sulfate as the cavitation fluid and lipid solubilizing agent.

A solution containing 5% w/v glycerol and 1% w/v sodium lauryl sulfate was filled into the cavitation bellows of the SonoPrep® device. Commercially available alcohol wipes were used for the experiment. Healthy, adult volunteers of ages 20-60 were enrolled in the study.

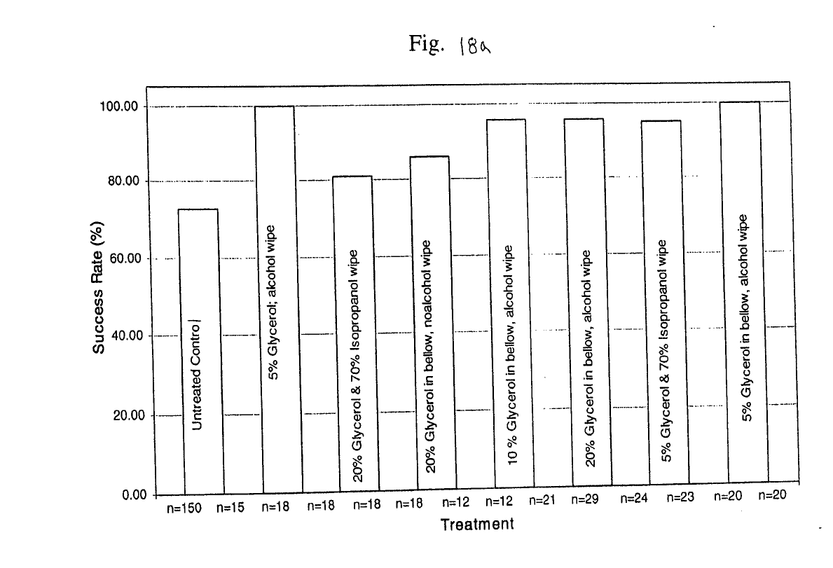

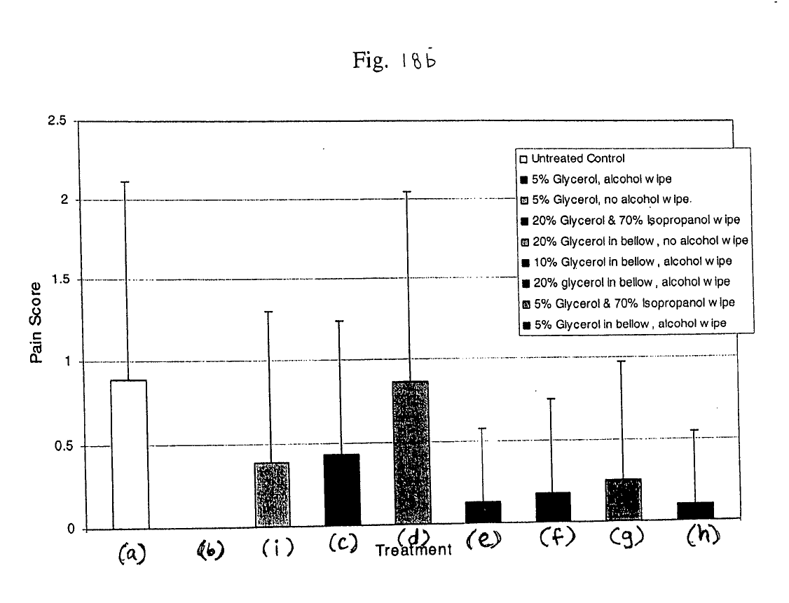

FIG. 18a is a graph of percent success rate of sonication by a SonoPrep® device obtained in human volunteers as a function of skin treatment. A successful sonication is one that achieves a skin impedance of 10 k-ohms and pain/discomfort scores <2. A non-successful sonication is one that has a pain/discomfort score >2. FIG. 18a shows data for various skin treatments including one or more of alcohol wipes (isopropanol), glycerol wipes, and ultrasonication with cavitation fluid containing sodium lauryl sulfate and glycerol (5% w/v) in the bellows of the SonoPrep® device. Specifically, the groups tested were (a) untreated control, (b) 70% isopropanol wipe followed by a 5% glycerol wipe, (c) a single wipe having 20% glycerol & 70% isopropanol in water, (d) 20% glycerol in bellows, no alcohol wipe, (e) 70% isopropanol wipe followed by 10% glycerol in bellows, (f) 70% isopropanol wipe followed by 20% glycerol in the bellows, (g) a single wipe having 5% glycerol & 70% isopropanol in water, and (h) a 70% isopropanol wipe followed by 5% glycerol in bellows. Data in FIG. 18a indicate that a skin pretreatment method of an isopropanol wipe followed by ultrasonication with cavitation fluid that contained sodium lauryl sulfate and 5% glycerol was highly effective in improving the percentage of successful sonications from 60-70% (untreated) to 100% (treated) subjects. Data in FIG. 18a indicate that a skin pretreatment method of a 70% isopropanol wipe followed by a 5% glycerol wipe was also highly effective in improving the percentage of successful sonications from 60-70% (untreated) to 100% (treated) subjects.

FIG. 18b is a graph of average pain/discomfort score associated with ultrasonication by a SonoPrep® device in human volunteers for the same groups shown in FIG. 18a with additional data for a 5% glycerol wipe without an alcohol wipe, which is denoted (i) on FIG. 18b. Data shown in FIG. 18b indicate that the skin pretreatment methods described above are effective in lowering discomfort associated with ultrasound-induced permeation of the skin. The untreated control had a significantly higher pain score.

### EXAMPLE 5

In this example, sonication parameters as a function of concentration of the hydrating agent, glycerol (5-100% w/v), were evaluated. This example compares a success rate and a pain score for pretreatment with an alcohol wipe followed by a glycerol wipe.

Varied concentrations of glycerol (5-100%) were used in the hydrating step, which followed treatment with an alcohol wipe. Healthy volunteers of ages 20-60 were enrolled in the study.

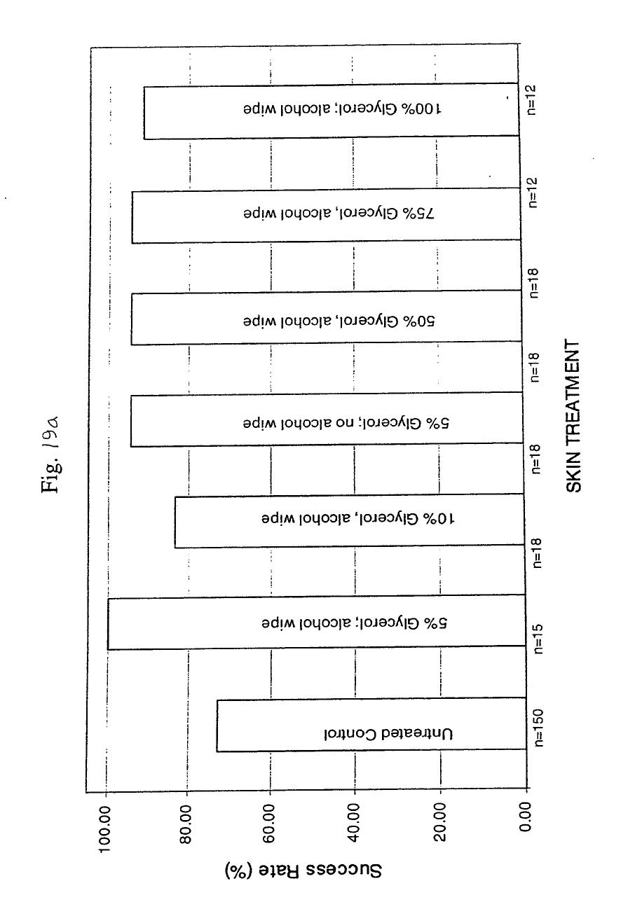

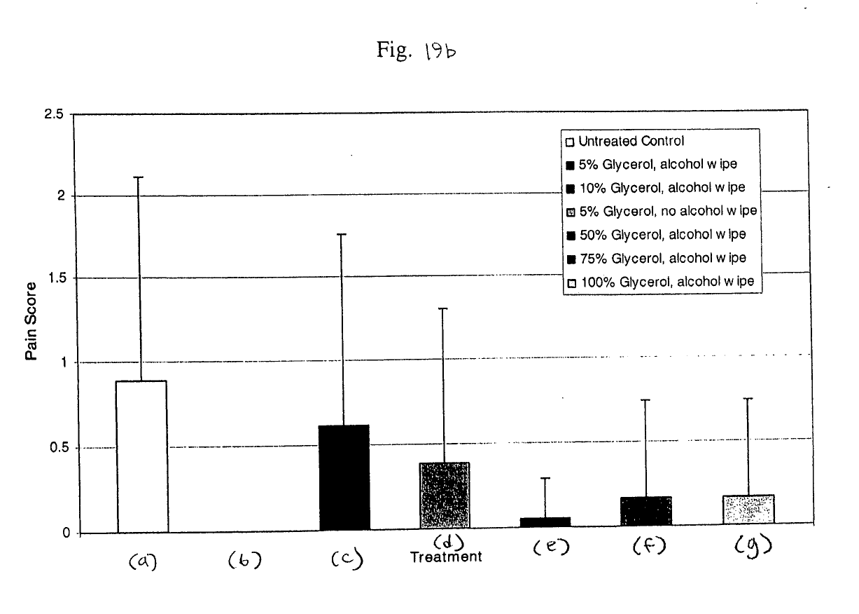

FIG. 19a is a graph of percent success rate of sonication by a SonoPrep® device obtained in human volunteers as a function of various skin treatment methods on the volar forearm. A successful sonication is one that achieves a skin impedance of 10 k-ohms and a pain/discomfort score <2. A non-successful sonication is one that has a pain/discomfort score >2. The groups tested were (a) untreated control, (b) 70% isopropanol wipe (alcohol wipe) followed by a 5% glycerol wipe, (c) 70% isopropanol wipe followed by a 10% glycerol wipe, (d) 5% glycerol wipe, no alcohol wipe, (e) 70% isopropanol wipe followed by a 50% glycerol wipe, (f) 70% isopropanol wipe followed by a 75% glycerol wipe, and (g) 70% isopropanol wipe followed by a 100% glycerol wipe. The data in FIG. 19a show that 5% w/v glycerol in the hydrating wipe is a desirable concentration as determined by the percentage of successful sonications (100% for alcohol wipe followed by 5% w/v glycerol wipe). FIG. 19b is a graph of an average pain/discomfort score associated with ultrasonication by a SonoPrep® device in human volunteers for the same groups shown in FIG. 19a. Data in FIG. 19b demonstrates that discomfort is minimal (pain score=0) when pretreatment involves use of an alcohol wipe followed by 5% glycerol wipe.

### EXAMPLE 6

This example provides a skin hydration treatment before, after, or both before and after sonication of the skin. In this example, the skin hydration step can be performed as a pre-treatment step prior to sonication by SonoPrep®, or as an added post-sonication step to enhance hydration of the stratum corneum. This example enables a prolonged skin hydration step prior to or after sonication and can be used to deliver additional hydration to the skin. The method of hydration may be as follows: (a) a hydrating skin pretreatment step followed by sonication and (b) a hydrating skin pretreatment, then sonication, followed by another hydration step to further enhance permeability. The hydration may be carried out either by a wipe or by holding the hydration agent on the target site in a circular foam reservoir for up to four hours. In another application of the post hydration step, holding a reservoir of the hydrating fluid allows analytes such as blood glucose to be extracted efficiently from the interstitial spaces of the sonicated skin. It is hypothesized that bioactive agents are delivered more efficiently through highly hydrated skin. This example has been demonstrated to work for extraction of serum glucose by holding a reservoir of fluid over the sonicated site.

### EXAMPLE 7

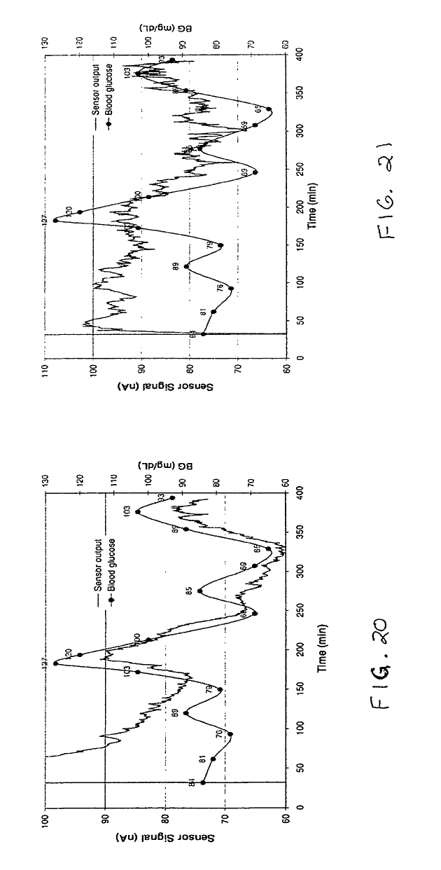

This example provides for skin hydration by contact with an electrolyte solution, isotonic solution, and/or an osmotic solution. The hydration agent can be an electrolyte solution (e.g., 0-1 M sodium chloride, 0-1 M potassium chloride or other biocompatible electrolytes) on the target skin site sealed in a circular foam ring for up to four hours. The presence of an electrolyte solution in the reservoir allows analytes to be extracted from interstitial spaces in the skin. In another embodiment, the hydration agent may comprise an isotonic solution (a solution having the same osmotic pressure as blood, such as phosphate buffered saline solution) on the target skin site, sealed in a circular foam ring for up to four hours. Additionally, in another embodiment, the hydration agent may comprise a compounded electrolyte solution which contains an osmotic agent such as 0-2 M lactic acid, an electrolyte such as 0-1 M sodium chloride or 0-1 M potassium chloride, a surfactant such as 0-1 M Triton X-100, Tween 80 or sodium lauryl sulfate, a pH buffer such as 0-1 M potassium phosphate, a transdermal enhancer such as 0-0.5 M glycerol, or any other biocompatible components on the target skin site, sealed in a circular foam ring for up to four hours. In FIG. 20, an example is shown with the following parameters for a hydration agent: 0.137 M sodium chloride, 0.0027 M potassium chloride, 0.01 M phosphate, 0.25 M lactic acid, pH=7. In this example, the period for skin hydration was 45 minutes before sensor application. As can be seen in FIG. 20, this example resulted in successful sensor response to fluctuations of blood glucose (BG). In addition, the sensor signal shows good correlation (r=0.83) with the changes of BG levels after 120 minutes of break-in period.

### EXAMPLE 8

This example describes a method to enhance transdermal transport during sonication by use of an applied vacuum or pressure. The use of vacuum or pressure may enhance extraction of interstitial and systemic analytes such as blood glucose. This method comprises application of a hydrating chamber combined with up to 50 psi vacuum or pressure on the target skin site for up to four hours. In one aspect, the hydrating chamber may have a reduced pressure relative to the ambient pressure. In another aspect, the hydrating chamber may have an increased pressure relative to the ambient pressure. The vacuum or pressure may be applied for a time sufficient to enhance transdermal transport. In one example, a vacuum of about 4 psig is applied to sonicated skin for a period of approximately 10 minutes. Application of a slight vacuum post-sonication according to this example can enhance diffusion of an analyte through the skin into a hydrating reservoir or into a detection device. In FIG. 21, an example is shown with one minute application of vacuum controlled between 15 to 20 kPa prior to sensor application. As can be seen by reference to FIG. 21, the sensor signal shows moderate correlation (r=0.73) to the changes of BG levels after a 120 minute break-in period.

### EXAMPLE 9

In this example, a skin hydration technique is utilized with Sonication prior to application of a local anesthetic. First, a skin site is prepared by application of an alcohol wipe followed by a glycerol wipe. Next, the skin site is subjected to ultrasound using a SonoPrep® device until the skin reaches a desired impedance. A solution of 4% lidocaine is applied topically to the sonicated skin site. Within a period of about 5 minutes after application of the lidocaine the local anesthetic will have taken effect and a subsequent operation requiring local anesthesia may be applied to the affected area proximate the skin site. In this example, hydration of the skin site prior to application of a local anesthetic will provide faster local anesthesia.

### EXAMPLE 10

In this example, a skin hydration technique is utilized with Sonication prior to application of a sensor for continuous transdermal glucose monitoring. First, a skin site of a patient is prepared by application of an alcohol wipe (70% isopropanol) followed by a glycerol wipe (5% glycerol). Next, the skin site is subjected to ultrasound using a SonoPrep® device until the skin reaches a desired impedance. The senor shown in FIG. 9 is then applied to the skin site. Finally, a continuous signal establishing the blood glucose concentration of the patient is established according to the operation of the sensor shown in FIG. 9. In this example, hydration of the skin site prior to sensor placement will provide improved blood glucose monitoring.

Other embodiments and uses of the invention will be apparent to those skilled in the art from consideration of the specification and practice of the invention disclosed herein. The techniques for enhancing transdermal transport described herein may be applied to any procedure that utilizes permeation of a biological membrane (e.g., skin). For instance, the delipidation and/or hydration products and techniques described herein may be applied to continuous transdermal blood glucose monitoring, transdermal drug delivery, electrophysiology, or any technology that entails increasing the porosity of a biological membrane. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A method for transporting a substance across a biological membrane comprising:
applying a delipidation agent to a portion of the biological membrane;
applying a hydration agent to the portion of the biological membrane;
sonicating the portion of the biological membrane; and
transporting the substance across the biological membrane.

2. The method of claim 1, wherein the step of applying the delipidation agent is carried out prior to the step of applying the hydration agent.

3. The method of claim 2, wherein the delipidation agent is applied using a wipe and the hydration agent is applied using a wipe.

4. The method of claim 2, wherein the delipidation agent is applied using an alcohol wipe and the hydration agent is applied using either a glycerol wipe or a baby wipe.

5. The method of claim 2, wherein the delipidation agent is applied using an isopropyl or ethyl alcohol wipe and the hydration agent is applied using a 5% w/v glycerol wipe.

6. The method of claim 2, wherein the delipidation agent and the hydration agent are applied using a baby wipe or a glycerol wipe.

7. The method of claim 2, wherein the delipidation agent is applied using a wipe and the hydration agent is applied during the step of sonicating.

8. The method of claim 7, wherein the delipidation agent comprises alcohol and the hydration agent is applied using a cavitation fluid comprising glycerol and sodium lauryl sulfate.

9. The method of claim 1, wherein the step of applying the delipidation agent is carried out simultaneously with the step of applying the hydration agent.

10. The method of claim 9, wherein the delipidation agent and hydration agent are the same.

11. The method of claim 1, wherein the delipidation agent and the hydration agent are different agents that are applied in combination.

12. The method claim 11, wherein the delipidation agent comprises an alcohol and the hydrating agent includes an amphiphilic molecule.

13. The method of claim 1, wherein the step of applying the hydration agent is carried out simultaneously with the step of sonicating the portion of the biological membrane.

14. The method of claim 13, wherein the step of sonication comprises contacting a coupling solution to the biological membrane.

15. The method of claim 14, wherein the coupling solution includes a component selected from the group consisting of water, saline, ethanol, isopropanol, sodium lauryl sulfate, Triton X-100, DMSO, linoleic acid, azone, polyethylene glycol, histamine, EDTA, sodium hydroxide, sodium octyl sulfate, N-tauroylsarcosine, octyltrimethyl ammoniumbromide, dodecyltrimethyl ammonium bromide, tetradecyltrimethyl ammoniumbromide, hexadecyltrimethyl ammoniumbromide, dodecylpyridinium chloride hydrate, SPAN 20, BRIJ 30, glycolic acid ethoxylate 4-ter-butyl phenyl ether, IGEPAL CO-210, and combinations thereof.

16. The method of claim 1, wherein the delipidation agent comprises alcohol and the hydration agent comprises glycerol.

17. The method of claim 16, wherein the alcohol comprises isopropyl alcohol.

18. The method of claim 1, wherein the delipidation agent is selected from the group consisting of tweens, polyethylene glycols, alcohols, micelle-forming amphiphilic polymers, pluronics, fatty acids, bile salts, cholesterols, cleansers, detergents, surfactants, and combinations thereof.

19. The method of claim 1, wherein the hydration agent is selected from the group consisting of tweens, polyethylene glycols, amphiphilic molecules, electrolytes, hyaluronic acids, glycerols, buffers, salines, osmotic agents, and combinations thereof.

20. The method of claim 18, wherein the hydration agent is selected from the group consisting of tweens, polyethylene glycols, amphiphilic molecules, electrolytes, hyaluronic acids, glycerols, buffers, salines, osmotic agents, and combinations thereof.

21. The method of claim 1, further comprising a step of applying a permeability enhancer to the portion of the biological membrane.

22. The method of claim 21, wherein the permeability enhancer is selected from the group consisting of aloe, histamine, soy lecithin, vitamin E acetate, and combinations thereof.

23. A method of transporting a substance across a biological membrane comprising:
providing a biological membrane exposed to a first pressure;
sonicating a portion of the biological membrane;
changing the pressure over the portion of the biological membrane to a second pressure after sonicating the portion of the biological membrane; and
transporting the substance across the biological membrane.

24. The method of claim 23, wherein the second pressure is less than the first pressure.

25. The method of claim 24, wherein the second pressure is about 4 psig lower than the first pressure.

26. The method of claim 25, wherein the first pressure is ambient pressure.

27. The method of claim 25, wherein the second pressure is maintained for about 10 minutes.

28. A method of preparing a biological membrane for transdermal transport of an agent comprising treating the biological membrane with a hydrating agent before, after, or both before and after sonicating the biological membrane.

29. The method of claim 28, wherein the hydrating agent comprises an electrolyte, isotonic agent, or an osmotic agent.

30. The method of claim 28, wherein the hydrating agent comprises sodium chloride, potassium chloride, phosphate buffered saline, lactic acid, Triton X-100, Tween, sodium lauryl sulfate, potassium phosphate, or glycerol.

31. A method of preparing a biological membrane for sonication comprising treating the biological membrane with a solution comprising alcohol, Lippo Gel, bile salt, or glycerol prior to sonication.

32. A method for sampling and analysis of an analyte from a body fluid in a patient comprising:
increasing a permeability level of an area of a biological membrane of the patient;
applying a transport force to the area to extract the analyte from the body fluid in the patient through the area and into a medium;
continuously reacting the analyte in the medium to produce an electrical signal at an electrode adjacent to the medium, the electrical signal representing a rate of reaction of the analyte; and
calculating an analyte concentration in the body fluid in the patient based on the electrical signal.

33. A system for sampling and analysis of an analyte from a body fluid in a patient comprising:
an ultrasonic applicator that applies ultrasound to an area of a biological membrane of the patient;
a medium comprising a substance that reacts with the analyte;
an electrode positioned adjacent to the medium, the electrode being adapted to receive an electrical signal produced by the reaction of the analyte with the substance in the medium, the electrical signal representing a rate of reaction of the analyte with the substance; and
a processor electrically connected to the electrode; wherein the processor is programmed to continuously calculate a concentration of the analyte in the body fluid of the patient based on the electrical signal.

34. A transdermal analyte monitoring system comprising:
a sensor body;
a medium supported by the sensor body and adapted to interface with a biological membrane, wherein the medium is adapted to prevent accumulation of analytes and analyte indicators during operation; and
an electrode adapted to detect the presence of an analyte within the medium.

35. A method for transdermal analyte monitoring comprising the steps of:
providing a sensor body, a medium and an electrode;
positioning the medium adjacent to the surface of a biological membrane; and
monitoring a flux of the analyte through the biological membrane using the electrode.

36. The method of claim 35, further comprising calculating the concentration of analyte within the body fluid opposite the biological membrane from the sensor body.

37. The method of claim 36, wherein the calculation provides a real-time measurement of analyte concentration within the body fluid.

38. The method of claim 35, wherein the rate of analyte consumed within the medium is about equal to a rate of analyte diffusing through the biological membrane into the medium.

39. A transdermal analyte monitoring system comprising:
a medium adapted to interface with a biological membrane and to receive an analyte from the biological membrane; and
an electrode assembly comprising a plurality of electrodes;
wherein the medium is adapted to react continuously with the analyte; and
wherein an electrical signal is detected by the electrode assembly, and the electrical signal correlates to an analyte value.

40. The transdermal analyte monitoring system of claim 39, wherein the analyte value is the flux of the analyte through the biological membrane.

41. The transdermal analyte monitoring system of claim 40, wherein the analyte value is the concentration of the analyte in a body fluid of a subject.

42. The transdermal analyte monitoring system of claim 39, further comprising a sensor body that supports the electrode assembly and the medium.

43. The transdermal analyte monitoring system of claim 39, wherein the analyte comprises glucose.

44. The transdermal analyte monitoring system of claim 39, wherein the medium comprises a hydrogel and glucose oxidase.

45. The transdermal analyte monitoring system of claim 39, wherein the medium comprises a hydrogel.

46. The transdermal analyte monitoring system of claim 39, wherein the biological membrane comprises skin.

47. The transdermal analyte monitoring system of claim 39, wherein the electrode assembly comprises a working electrode, a counter electrode, and a reference electrode.

48. A transdermal analyte monitoring system comprising:
a medium adapted to interface with a biological membrane and to receive an analyte from the biological membrane; and
a sensor comprising an electrode assembly, the electrode assembly comprising a plurality of electrodes;
wherein the medium is adapted to react continuously with the analyte, an electrical signal is detected by the electrode assembly, and the electrical signal correlates to an analyte value.

49. The transdermal analyte monitoring system of claim 48, wherein the analyte comprises glucose.

50. The transdermal analyte monitoring system of claim 48, wherein the medium comprises a hydrogel and glucose oxidase.

51. A method for monitoring an analyte comprising:
positioning a medium with respect to a biological membrane such that the medium can receive an analyte from the biological membrane;
coupling an electrode assembly to the medium, the electrode assembly comprising a plurality of electrodes; and
continuously reacting the analyte with the medium;
wherein an electrical signal is detected by the electrode assembly, and the electrical signal correlates to an analyte value.

52. The method of claim 51, further comprising pretreating the biological membrane to increase a permeability of the biological membrane.

53. The method of claim 51, wherein the pretreating step comprises applying low frequency ultrasound to the biological membrane.

54. A cartridge for use with a sonication device comprising:
a cartridge body adapted for insertion into the sonicating device; and
a chamber within the cartridge body, wherein the chamber contains cavitation fluid comprising a hydrating agent.

55. The cartridge of claim 54, wherein the cavitation fluid further comprises a lipid solubilizing agent.

56. The cartridge of claim 55, wherein the cavitation fluid comprises glycerin and an alcohol.

57. The cartridge of claim 54, wherein the hydrating agent is selected from the group consisting of tweens, polyethylene glycols, amphiphilic molecules, electrolytes, hyaluronic acids, glycerols, buffers, salines, osmotic agents, and combinations thereof.

58. The cartridge of claim 55, wherein the hydrating agent is selected from the group consisting of tweens, polyethylene glycols, amphiphilic molecules, electrolytes, hyaluronic acids, glycerols, buffers, salines, osmotic agents, and combinations thereof.

59. The cartridge of claim 55, wherein the lipid solubilizing agent is selected from the group consisting of tweens, polyethylene glycols, alcohols, micelle-forming amphiphilic polymers, pluronics, fatty acids, bile salts, cholesterols, cleansers, detergents, surfactants, and combinations thereof.

60. The cartridge of claim 58, wherein the lipid solubilizing agent is selected from the group consisting of tweens, polyethylene glycols, alcohols, micelle-forming amphiphilic polymers, pluronics, fatty acids, bile salts, cholesterols, cleansers, detergents, surfactants, and combinations thereof.

61. A kit for use with a sonication device comprising:
an ultrasonic coupling medium cartridge adapted to interface with the sonication device; and
a skin preparation pad, wherein the skin preparation pad comprises a hydrating agent.

62. The kit of claim 61, further comprising:
a disinfectant cartridge adapted to interface with the sonication device;
one or more target rings; and
an injection site marker.

63. The cartridge of claim 61, wherein the cavitation fluid further comprises a lipid solubilizing agent.

64. The cartridge of claim 63, wherein the cavitation fluid comprises glycerin and an alcohol.

65. The cartridge of claim 61, wherein the hydrating agent is selected from the group consisting of tweens, polyethylene glycols, amphiphilic molecules, electrolytes, hyaluronic acids, glycerols, buffers, salines, osmotic agents, and combinations thereof.

66. The cartridge of claim 63, wherein the hydrating agent is selected from the group consisting of tweens, polyethylene glycols, amphiphilic molecules, electrolytes, hyaluronic acids, glycerols, buffers, salines, osmotic agents, and combinations thereof.

67. The cartridge of claim 63, wherein the lipid solubilizing agent is selected from the group consisting of tweens, polyethylene glycols, alcohols, micelle-forming amphiphilic polymers, pluronics, fatty acids, bile salts, cholesterols, cleansers, detergents, surfactants, and combinations thereof.

68. The cartridge of claim 66, wherein the lipid solubilizing agent is selected from the group consisting of tweens, polyethylene glycols, alcohols, micelle-forming amphiphilic polymers, pluronics, fatty acids, bile salts, cholesterols, cleansers, detergents, surfactants, and combinations thereof.
